SEQUENCE LISTING (27 sequences)

SEQ ID NO: 1 - DNA, 1950 bp, Dreissena bugensis
atggtgcttaaggttgaagcagacacgcttacacataaccatgccattctaccttggattggtcaggggtttggtagcgtaaacactggggccttccacaagatgtctcagttgcacttccagaccaatacaccaccaaaccaaccaatgtcacaagagacctttgattacctctggaataccctcgaagaggtcactgatcatggcgactacacccacatcaacgctagggagttgtcatacacatacgatgacagtgatgaaagtacatctatgcaagtggaaaagttcaaaatctcccaccaagatgtctcagacttgttgaacccaatcatcggcaccacctcctcctcatccatgtccccggactctcaaacaaacatcagcggctccactgcttcctccccttaccatgaaatggcactcacaagtccccctccatacagtccacataccaacatgacatcacccatccccacagtaccatcaaacaccaattacccaggagattatggctttgaaatttccttcgccacaccatcaaaagagaccaaatcgaccacatggacatattcagagacgctgaaaaagctttatgtgcgcatggcaaccacttgtccggtgaggttcaagaccgcccgccccgcaccccagggcgccttcatcagaaccatgccaatcttcatgaagcccgagcacgtacaggaccccgtcaaacggtgccctaatcacgccacctcaaaagagttcaacgagaatcaccctgcccccaatcacctggtgcgctgcgagcacaagctggccaagtacgtggaggatgtgtgtacatgccgccagtcggtgatcatcccccaggagataccccaggccgggtcagagtgggtcaccaacctcttccagttcatgtgcctcggctcgtgtgttggagggcccaacaggcgacccctgcagatcatcttcaccctcgagaaagacaaccaggttctgggccggagatgtgtggaagttcgtatctgtgcctgcccaggtcgtgatcgtaaggcagatgagaaaggcatgttacccgttgtacccggaatgaagaaaaattttcagaagatcaccatgggaacagaaatgaccacaattacatctggcaagaaaagaaaattggatgacgatgaaacattcactttgacggtaatacggggcaaagaaaattacgacatgctttgtaagatcagggacagccttgaaatagccgcacaagtcccacagaaccttgtacaaaactataagcaacgccaagtggaggttcaaagacaggactctgcaggcaccagttcttcccgtcaggtctccatggcaaccgcacagtctacaagtcgcacgctctcgcagacaacgctcactgctgatgggaaggcgcacaccctgccgttcaattccaatgaaagcgcatctcatatccgactgtacattcaggtgcaggtgaccagttctgatgtgagtcatgatggttccaatggcgtaccacagccagtcaaagaagagaccgtgatgcatgaggacaataccatccaaacgtggttaaatgccatcggactgggtgcatacatcgacactttccaggaacagaacttatacagtgttctacagcttgacaatttctctctggaggatctggccaagatgaagatcggcaacgctcaccgcaacaagatctggcagagcatcctggacctgcgcagcggcggcttcacagacggcacaacccaggagcacctgctaaccacacagggctccaccgcgtccacgatcagcgtgcagagctccatctctcagaacagcacgtacaacccgggcttctacgaggtcacgcgctacacgttcaagcataccatctcgctgacgaaagaggacaggcatccgggtgaatctgttctatatgaaagaaaaaaggaccactag

SEQ ID NO: 2 - protein, 649 aa, Dreissena bugensis
MVLKVEADTLTHNHAILPWIGQGFGSVNTGAFHKMSQLHFQTNTPPNQPMSQETFDYLWNTLEEVTDHGDYTHINARELSYTYDDSDESTSMQVEKFKISHQDVSDLLNPIIGTTSSSSMSPDSQTNISGSTASSPYHEMALTSPPPYSPHTNMTSPIPTVPSNTNYPGDYGFEISFATPSKETKSTTWTYSETLKKLYVRMATTCPVRFKTARPAPQGAFIRTMPIFMKPEHVQDPVKRCPNHATSKEFNENHPAPNHLVRCEHKLAKYVEDVCTCRQSVIIPQEIPQAGSEWVTNLFQFMCLGSCVGGPNRRPLQIIFTLEKDNQVLGRRCVEVRICACPGRDRKADEKGMLPVVPGMKKNFQKITMGTEMTTITSGKKRKLDDDETFTLTVIRGKENYDMLCKIRDSLEIAAQVPQNLVQNYKQRQVEVQRQDSAGTSSSRQVSMATAQSTSRTLSQTTLTADGKAHTLPFNSNESASHIRLYIQVQVTSSDVSHDGSNGVPQPVKEETVMHEDNTIQTWLNAIGLGAYIDTFQEQNLYSVLQLDNFSLEDLAKMKIGNAHRNKIWQSILDLRSGGFTDGTTQEHLLTTQGSTASTISVQSSISQNSTYNPGFYEVTRYTFKHTISLTKEDRHPGESVLYERKKDH

SEQ ID NO: 3 - DNA, 7086 bp, Dreissena bugensis
atgtctctgggccaagtgttgtctggaatgaagaccaaaaatgttcagtggctacaaccagtggattgtaaccactgcagactggatctcatgtccaggcttgttcactggattgtcacgcagttcgtgtttatccttctgcggacgcacttctacgtcactgacacaaccttcctacgataccgactggtttactacagacaggccacttggacaaggctgcacattcagggattgactgtcctacctgtcagtgaatcagcagctcaaggatctccacaaggtcctcacacatctaaaggactgcagaccctcgctcatcggatcggggaagagttgcaagaagagtgtgtggttcgtaagtatttcacctttgccctgaaaggaggccatgtgcgcaagacattccaaagacacgcagcccacctcaccgacttcatgcaggatttctcaaattttgcaaaatcacaggtcgatgagaagaatttgcagggtgttgtccttgtggataaggtcttctaccagcacttgtcagctgagacagtactaacaattcttaggagccacatttacaacaatattgtgaaggtaggcaggcagtattacctccaaaaggagggaatcagtcagggctctgttctctccaccctgctctgtaacttctactatgcgtgcatggagcgtgaccacatcgccacgcaacgagaggagctgcttatgagggtcgtcgatgactacctctttgtgacgccctccttggaacgggcaaccagcttcctccggaccatgctagctgggatcaagggctacaattgttataccaatgtagacaaagtcatgagtaatttcccttgcaatgtgacagaaaaccagtccattgagtacattgaaacagccctttgtagtaagagttacttggtcagagtgttaattggtacaatggttgggaatggtatggatggcatggcagtccagcggacaagatgggcacagcaagggttcgcaattggtaagggacctgaagtaccctgctgctcctctggaagtcagaccgtacaagcacaaaaggtaaccgaatatcagctccgaatggataagagtctagcaaaaaaactatctgcagctagacgtacaaaacatttggacttcgaaataaagagcggcaatgttgtaatcgtcgctgatctggctaccttcgaactgcttaaggaagctgctctgtatttctataaaaatgatacatcagtcaaagatgatacagttatccaatcgacagtcgataagaaaaatagttcagtttcatatgccataaaatataaattctacactattaacatttatgcaacttcaagtaaattcatggtaaatggacagaatacacagttgtttgtagggcagcacctttcagcgatacagaacattgtaaggcatgctactgtgaatggacaacctgtaaacctagacagtgtgaatcaactgttggcagaaagtatccagagtgcactgctcaactcttcaatagcaactgaacaatcaagtaaggcaaaaacttctatttcttcaaaaatcacaatgaatactaaggcgaagagtactgaaagtaaacctaccaatggtgatgaacctgaagttaaatgtttcaagtgtaatagaactctaaaaactggtgttgaatgtacagtaaatcataagaatgaatctcattggatacattatggatgcttgaatttgaacgacactgaacttgaacttgttaaaaaccaacctgaagatgctccatacacatgtacattgtgtgccaagcatgataatacaaggactctacctgtaaaaataacgacaaaccgttctacgttaaacattcctgctctaccaaatatacccgtctcaaatgcatttgccattcttaccgaggaacagattaacactgaacctgttctttacattgaacaatgctatatctgttgtaaggacaaacaacaggatcaagtctgtgaaacttgtcaaggtatttgtcatcatgattgtgttgtgtttaacactgacaatattacagcttcctgcttggcatgtgtaggggaaaatcagcagctaaatctggcaggagagcagtctgtcgatagcaacccgcaggtacaggggaaccagctggttagtacagacacaggtgtgattgctatccaagacgaaaaacgagctctactaaaaggtttgaatgaaaaagacaaagagattaggaaattgcaaaatgatttaaagttaaaagataaagaactatcagaagcatacaagcataagccaaaaactgacatatatattaaaaaacatgaagctaaatgtgaagaacaagaaaggacaattaaaactctattgaataaaattgagttcttggaaaacaaagttgaatcccttgagaaaactgctaaatattcgaaccacccaacacagtctaatggtacatccgaaaacaatgagcttgcagcttcaattcacagtcaagtgacaagtctagttctaaaaaaagtgagtcaagaattggaaagctttggttcacaatggcttgcatcaagtaacaccgctaataaatctagtacacaaacacaaccaactaccacaagcattgagaatcagcagactgctcaagaaactattccaaaccaaaacttaaatgaaaatacagtatactccaatgttatgaaaaatgctaacactacaggtcaaagagatgggcggactcatactggtgtgcctcagtcaacaggcagtgtgacgaaggttgaaggtgacgtcatactcccagaaggattcgtggcattttcaaggaaccagctccacgctgtgatgcagggacgacctatactaaagtctcgttcgtacaaacctccaagcaacactcagtataacacaaacagtaacaagagtaattcacgtctgacactgtccaagacagttcagcataacattaacaactatggcaacataataactgaaccacaaaacagaactgtaaaagtcgataaagttattcaacaggacttaaagctgaaatctagtaaaactgaccctgatattgaaactatcgaaccatctaatactgacaaaagtgaacaacctgatagccaaccagctgaactgtccgatgcaaacaaacctattgtaacccaaacgattgacctaacaaaagggcaagcaatagacaaaaatcatttttgtagccgagcgccaggatttcaaccaaagaagatagcacaaaacgaagacactaactcagacactataaatctggtcgcatacaactgcaagaatgttaaaactagtgttacatgcatcaatgaactatttaaaactgctcaactcatattgttatctgaaacatggctttatgaatatgaactttatctcttgaatgaaataggaagtaatatctgctcagcaggaaaatgcagtgatttctacaatccagtacctcctggacaacgtaacaggggccacgctggcgtagcagtgttatggaggaaagaaattaatcacctcattacagagctacccgacggaaatgagagactccagtgtttggagctagcattagaacatacaaaatatctgatagtagctgcatatctcccaacgacaggtggacttgaaactgaaacagaatttctagaatgtgttgacatcttaagagaaataattcttcaatatcaaaactcgcatgacatcattacaggaggtgacctgaacgttgatctgtcggtgaacggtccagttctaaaacgaacagtttacatcaaacatatgatagacgaactgaatttaaaatacgactgcagtggaaaaactttcattaactctcttggcagggaagtaagtgaactagattactttcttgtgaaactcagaaatacaaacagtaagtccatgggaaaaattgtgttgaaccaactcgactctaatgtatctgatcaccacccagttcaaatatcggtaaaagttaataaactaattcttttgaaaaacaaagttcaaaaatctgtaaaaaagtttaaagttacatgggacaaaatcaatcaggaatcatttgcagaagacatgagaaaaggtgtattaatgcttaaaccaaatgaacttaaagaaaatcagaatatcgaagtttttgctcagaacattatgtctgttatgaaagacactatgaatctgcactcaagtgaaataaaacatatgaactccaaacctaaactcaaagtctggacgcaagacattggaatcgctcttcgagacaagcctgaagcgtataagaaatgggctaccgaaggcaaaccgcaagatccaaataaccagttactctctaacgtcaaaaccacaaagagactctttcgtagttcaatccgctgtgagcaagccagaagaacaatgcaagaccgtgacaaaattttgaactctagagcccaagatcctaagacatttcataaacttatcaataaaaatagaaagaataagaattgtttcattgaagacttaaatgtagatggtgaatctttcacaggagtcgacaaggtagctgatggtttcaaaaagcatttcaaagcgctagcgggacattcagaaaacccaaatttttacaatgaatatcacaaaacagtaatcaaagagtgtgaccacatcagcgatatggtttttgaaacagaagtacagccaattactcctaaggaacttcaaagggcaatacggggaataaaccgcgggaaagcacctgactaccatgggttatctattgaatgtgtaataaatggaggccctattctacaaaatgtgattttgaaacttttgaattctatcatgttatctggatatgtgcctgagtgtatgaaaattggtattttaacaccgatatacaaaaataagggttccaggaacgacgcttgtaacttccgaggaattactgtgctacctgtcatagaaaagattcttgaaatggttctgaaaatacgacttgttccaactcttgaaaaagaccagagtgctttccaacgggggttcactgccaaaacctcaccacttcacgcagcgttgatagtcgaagaggtttcccgcgagtataaagataagggagaggacattgatcttgttttccttgacgcaaaagccgctttcgatgttgtggatcatcatcacctactacgcaggttataccactctggcgttaatgatagacattggacaatttttaaaagtattcatacgcagtcgactagtgttgtgaaatgggcaaattcaagattagacccctttgaggttctacagggtgtgagacaaggagggatcaccagtactgacctatacaaaatttacatcaaccctctgctgaaacgtttagagatggcggaagaaggatgtgttataggtgatgttaggtgcaacactagtgcatgtgcagatgatgttacattgaattctgaaaaaccaggagaaacatcagttcttataagtatggccgaaacatttgccaactacgaaagatatattttacagcctaaaaagacggaggctctcaaagtggttacaagttctagaaaagaaatagttgaagaagattttgaaatatatggaaataaaattacaaacaccgatacttgtactcatctaggtttgaaacgttctcacactatcagctctacagcggaacaaaatgttgataacaatattcaaaaagcacgaagaacagtatatagctttatgtcgtcaggtttctatggtgctggtggtttggacgtcccatcaatcctacatattttggaaatgcatatcattcccattctgttatatggactagaaattattttacctaaaaagacacaaattgataagctcgaaatttttcaaaaacgaatactaaagcaactacttgtgttacctagtagtacacctgacattgcaatctactcaataagtggtctccttccagtaaaaatccaaatacacaaaagagctttaatcatgtataacaatgtgtgcctacaaagtgaaagtgcagtggaacgttgtatagctgttagacagttgactgtaaagagcaacaagagcgctagttggtttgtagacatccgtaagttattctggctgtatgaacttggcgatccagaagatttgcttgagtctccaacagagaaggaacactggaagagaacagttaacagaacaatagacaatatatggttacaacagacagttgctgaatcaaaaacaatgaaaactttgcaaaatcttaacatgaacatggttaagccaagaaaaccacaccctcttctacaacaacaggcggtctctacatacgatgcgaaccggcaagtactcaaactcaagtttatgtgtggcaagtacattttgcagagtgaccgggcatcctttagtaaaaacaaggtagatgacacatgcagagtgtgctacaagtctcctgagacacttcaacacatggtcctcgaatgttcgggcttgtctgcggtaagagatcctatcctccgtgacatagagagtgaaatcgaaaaatccttcccatgtctgtggcaatggtacacacaagatcagaaagtaactgctctcattgattgtactgttctttacaaaaagcacaaactttccaaagtagaatgccagagacttcacaaaattgattttcaatgccggcgtctattttttacattgcatacgaaacgctttaaaattttcgagttgtcgaaaggtattacagacaccatgacttttgactttgttagggaacctggtaagacattcaaacggaaattgattgggctgtgttggaaagcgttcctggtgacgtattgtcgacggcatatcttcccgcttctcacaaaacgggtcaagactggactagtgactgtgaaaaagcacttgaaccagtacttggtgtctttgtcgaaagaacgctaccacagtgtgaatcgaacccgtgacctcccggtcgctaggcggacaccatatccgttacaccacggcgaccttattaattccagttcaaccgtcccagaccatacttggcaattgctgagcgcgactcgtatcttatattga

SEQ ID NO: 4 - protein, 2361 aa, Dreissena bugensis
MSLGQVLSGMKTKNVQWLQPVDCNHCRLDLMSRLVHWIVTQFVFILLRTHFYVTDTTFLRYRLVYYRQATWTRLHIQGLTVLPVSESAAQGSPQGPHTSKGLQTLAHRIGEELQEECVVRKYFTFALKGGHVRKTFQRHAAHLTDFMQDFSNFAKSQVDEKNLQGVVLVDKVFYQHLSAETVLTILRSHIYNNIVKVGRQYYLQKEGISQGSVLSTLLCNFYYACMERDHIATQREELLMRVVDDYLFVTPSLERATSFLRTMLAGIKGYNCYTNVDKVMSNFPCNVTENQSIEYIETALCSKSYLVRVLIGTMVGNGMDGMAVQRTRWAQQGFAIGKGPEVPCCSSGSQTVQAQKVTEYQLRMDKSLAKKLSAARRTKHLDFEIKSGNVVIVADLATFELLKEAALYFYKNDTSVKDDTVIQSTVDKKNSSVSYAIKYKFYTINIYATSSKFMVNGQNTQLFVGQHLSAIQNIVRHATVNGQPVNLDSVNQLLAESIQSALLNSSIATEQSSKAKTSISSKITMNTKAKSTESKPTNGDEPEVKCFKCNRTLKTGVECTVNHKNESHWIHYGCLNLNDTELELVKNQPEDAPYTCTLCAKHDNTRTLPVKITTNRSTLNIPALPNIPVSNAFAILTEEQINTEPVLYIEQCYICCKDKQQDQVCETCQGICHHDCVVFNTDNITASCLACVGENQQLNLAGEQSVDSNPQVQGNQLVSTDTGVIAIQDEKRALLKGLNEKDKEIRKLQNDLKLKDKELSEAYKHKPKTDIYIKKHEAKCEEQERTIKTLLNKIEFLENKVESLEKTAKYSNHPTQSNGTSENNELAASIHSQVTSLVLKKVSQELESFGSQWLASSNTANKSSTQTQPTTTSIENQQTAQETIPNQNLNENTVYSNVMKNANTTGQRDGRTHTGVPQSTGSVTKVEGDVILPEGFVAFSRNQLHAVMQGRPILKSRSYKPPSNTQYNTNSNKSNSRLTLSKTVQHNINNYGNIITEPQNRTVKVDKVIQQDLKLKSSKTDPDIETIEPSNTDKSEQPDSQPAELSDANKPIVTQTIDLTKGQAIDKNHFCSRAPGFQPKKIAQNEDTNSDTINLVAYNCKNVKTSVTCINELFKTAQLILLSETWLYEYELYLLNEIGSNICSAGKCSDFYNPVPPGQRNRGHAGVAVLWRKEINHLITELPDGNERLQCLELALEHTKYLIVAAYLPTTGGLETETEFLECVDILREIILQYQNSHDIITGGDLNVDLSVNGPVLKRTVYIKHMIDELNLKYDCSGKTFINSLGREVSELDYFLVKLRNTNSKSMGKIVLNQLDSNVSDHHPVQISVKVNKLILLKNKVQKSVKKFKVTWDKINQESFAEDMRKGVLMLKPNELKENQNIEVFAQNIMSVMKDTMNLHSSEIKHMNSKPKLKVWTQDIGIALRDKPEAYKKWATEGKPQDPNNQLLSNVKTTKRLFRSSIRCEQARRTMQDRDKILNSRAQDPKTFHKLINKNRKNKNCFIEDLNVDGESFTGVDKVADGFKKHFKALAGHSENPNFYNEYHKTVIKECDHISDMVFETEVQPITPKELQRAIRGINRGKAPDYHGLSIECVINGGPILQNVILKLLNSIMLSGYVPECMKIGILTPIYKNKGSRNDACNFRGITVLPVIEKILEMVLKIRLVPTLEKDQSAFQRGFTAKTSPLHAALIVEEVSREYKDKGEDIDLVFLDAKAAFDVVDHHHLLRRLYHSGVNDRHWTIFKSIHTQSTSVVKWANSRLDPFEVLQGVRQGGITSTDLYKIYINPLLKRLEMAEEGCVIGDVRCNTSACADDVTLNSEKPGETSVLISMAETFANYERYILQPKKTEALKVVTSSRKEIVEEDFEIYGNKITNTDTCTHLGLKRSHTISSTAEQNVDNNIQKARRTVYSFMSSGFYGAGGLDVPSILHILEMHIIPILLYGLEIILPKKTQIDKLEIFQKRILKQLLVLPSSTPDIAIYSISGLLPVKIQIHKRALIMYNNVCLQSESAVERCIAVRQLTVKSNKSASWFVDIRKLFWLYELGDPEDLLESPTEKEHWKRTVNRTIDNIWLQQTVAESKTMKTLQNLNMNMVKPRKPHPLLQQQAVSTYDANRQVLKLKFMCGKYILQSDRASFSKNKVDDTCRVCYKSPETLQHMVLECSGLSAVRDPILRDIESEIEKSFPCLWQWYTQDQKVTALIDCTVLYKKHKLSKVECQRLHKIDFQCRRLFFTLHTKRFKIFELSKGITDTMTFDFVREPGKTFKRKLIGLCWKAFLVTYCRRHIFPLLTKRVKTGLVTVKKHLNQYLVSLSKERYHSVNRTRDLPVARRTPYPLHHGDLINSSSTVPDHTWQLLSATRILY

SEQ ID NO: 5 - DNA, 7089 bp, Artificial sequence
  Synthetic nucleotide sequence that encodes Dreissena bugensis (quagga mussel) TERT (as shown in SEQ ID NO 4), but which has been codon optimized for expression by removal of codons that are not expressed well in dreissenid mussels
atgtctctcggtcaagtcctctctgggatgaagacaaagaatgtccagtggctgcagcctgttgactgcaaccactgccggcttgaccttatgtctcgactcgtacattggatcgtcacacaattcgtgtttatcttgctccggacacatttctatgtgaccgatactacattcttgcgatatcggctggtttactatcggcaagcaacatggactcgactgcacatccagggcctgactgttcttcctgttagtgaatccgcagcacaagggagtcctcagggacctcacacttcaaagggccttcaaacccttgctcatcgaataggggaagagctccaagaggaatgcgtcgttcgcaagtatttcacttttgcactcaagggtggtcatgttcgcaaaacttttcagaggcatgcagctcaccttactgacttcatgcaagacttctccaattttgccaaaagtcaagtggacgagaaaaaccttcaaggtgtggttctcgtcgataaagtattttaccaacacctttccgcagagacggtactgactattcttcgctcacacatttacaacaatatagttaaggtcggcagacaatattacctgcagaaggagggaataagtcaaggatctgtccttagtactctgctctgcaatttttattacgcatgcatggagcgcgaccatattgctacccaacgggaagaactgctgatgcgggtagtagacgactatttgtttgtaactcccagtctggagagggccacctcctttcttaggactatgctcgccggaattaaagggtataactgctacactaacgttgataaagttatgagtaactttccatgcaacgttactgagaaccagtctatcgagtatattgagactgctttgtgcagtaagtcctacctggtaagggtattgatcggcactatggtaggcaatgggatggatggtatggctgtccagcgcacacgatgggctcagcaggggttcgctatcggtaaaggccctgaggtcccatgttgctccagtgggtctcaaactgttcaagctcaaaaagtcaccgagtatcagctgaggatggacaaatctctcgcaaaaaagctctctgcagctaggcgcactaagcatcttgacttcgagatcaaatcaggcaacgtcgttatagtcgcagacctcgccacattcgagttgctcaaagaagctgctctgtatttctataagaatgatacaagtgttaaagacgacactgtgattcagtccaccgttgataaaaaaaacagtagtgtctcctacgctattaaatataaattctataccattaacatctacgccacttcctctaaatttatggtaaacggccagaatacacagctcttcgtaggacagcacctctccgcaatccagaatatcgttaggcacgcaaccgtgaacgggcagcccgttaacctggattccgttaaccagcttttggccgagtctatccagagtgcattgcttaacagttccatcgctactgaacagtcttccaaagcaaaaactagtatcagtagtaagatcacaatgaacacaaaagccaagagtaccgaaagtaagcctaccaacggtgacgagccagaagtaaaatgttttaagtgcaatagaacacttaagaccggggtcgagtgtaccgtaaaccacaagaatgagagtcactggatacattatggctgtctgaacctgaacgataccgagctggaattggtcaagaatcagcctgaggacgctccatatacatgtacattgtgcgctaaacacgataacactcggaccctgcccgtaaagatcacaactaatcgctcaacactgaacatcccagctctgcctaacatacccgtctccaacgcattcgcaatcctgactgaggaacaaatcaatacagaaccagtgctttatattgaacagtgctacatatgctgcaaagacaagcagcaagaccaagtatgtgaaacatgtcaaggaatctgccatcacgattgcgtcgttttcaacacagataatataacagcatcctgcttggcatgcgtcggcgaaaaccagcagctcaacctggctggcgagcaatcagtcgactccaacccacaagtccagggtaatcaactcgtatctaccgacactggagttatcgctatccaggatgaaaagcgagcacttcttaaggggcttaacgaaaaggacaaggaaatccgaaagctgcagaatgatctcaagcttaaagataaagaacttagtgaggcctacaagcataaaccaaagaccgacatatacataaaaaaacacgaagcaaagtgcgaagagcaggagcgcactataaaaaccctccttaacaagatcgagttccttgaaaacaaggtggaaagtttggaaaaaactgccaaatacagtaaccacccaacccagtcaaacgggactagtgaaaacaatgagcttgctgcatcaatacatagtcaggtcactagtcttgttttgaagaaggttagtcaagagttggagtcttttgggtctcaatggctcgcctcctcaaacacagctaacaaatccagtactcaaactcaaccaacaactacatctattgaaaaccagcagaccgctcaagaaactatccccaatcaaaatctgaatgagaacacagtttacagtaacgttatgaaaaatgccaatactactggccagcgcgacgggaggacacacacaggcgtgccccagagtaccggcagtgttacaaaggtggagggtgacgtaatactccctgaaggctttgtggccttcagtcgcaaccaactccacgcagtgatgcaaggcagacctatacttaagtcacggagttacaaacccccttctaatactcaatataatacaaatagtaataaaagtaacagtcggctcactttgagtaagacagtgcaacataatattaacaactacggaaatattataacagaacctcaaaaccgcactgtcaaagtggacaaggtaattcagcaggacctcaaacttaaaagttccaaaaccgaccctgacatcgaaactatagaacccagtaacacagataagtcagagcaacctgactctcagccagccgagctttcagacgccaataaacctattgtgactcagacaattgacctcacaaagggtcaagcaatagataagaaccatttctgctctagagctccaggtttccagcccaaaaaaatagcacagaacgaggacaccaactccgatacaatcaatctcgttgcatacaactgcaagaatgtgaagactagtgtcacatgtatcaacgagctcttcaaaactgctcaactgatattgctcagtgaaacatggctctacgagtacgaactttaccttttgaatgaaataggatctaatatttgctcagccggtaagtgtagtgatttttacaaccctgtccccccaggacaacgaaatcgaggtcatgctggagtggctgtactttggagaaaggaaataaaccatctcattactgaattgcctgacggaaatgaacggttgcagtgcctggaattggcacttgaacatacaaaatatttgatagtggccgcatacttgccaacaaccggcggtctggagaccgaaactgagtttcttgaatgtgttgatatactgcgggaaatcattctgcaatatcagaacagtcacgacattattaccggtggcgaccttaacgtagatttgagtgtgaacgggccagtgcttaagaggaccgtttatattaaacatatgatcgacgagcttaacctcaagtatgactgttcagggaagacctttatcaattctcttggacgggaggtcagtgagcttgactacttcttggtcaagctcagaaatacaaattcaaagtcaatgggtaagatagtcctgaatcagttggacagtaacgtttctgatcaccatcccgttcagatttcagttaaagttaacaagctgatcctgttgaagaacaaggttcaaaaatctgttaagaaatttaaagtaacatgggataaaattaatcaagaaagttttgctgaggacatgaggaagggtgttctgatgttgaaacccaatgaacttaaggagaaccagaacatagaagtatttgctcaaaatataatgtccgtgatgaaggatacaatgaatctgcattcctccgagattaagcacatgaacagtaaacccaaactgaaagtctggacccaagacataggaatcgcattgagagataagcccgaagcatacaaaaaatgggccaccgaggggaaacctcaggatccaaacaaccaacttttgtcaaacgtcaaaacaacaaagagactctttagatcaagtatcaggtgcgagcaagcacggcggacaatgcaagatagagataaaattttgaactctcgggctcaggatcccaaaacctttcacaagcttattaacaaaaacaggaagaacaagaattgtttcatcgaggatcttaatgtcgatggggagtcatttactggcgtagataaagtggctgatggcttcaagaaacatttcaaggcactcgctggacactcagaaaacccaaacttttacaatgaatatcataaaaccgtgataaaagaatgtgatcatataagtgatatggtatttgagactgaagtacagcctataacacctaaggagctgcaacgcgcaataagaggaattaatcgggggaaggctcctgattaccatgggttgagtatagaatgcgtaataaatgggggacctatattgcagaatgttatcctcaaactccttaattccatcatgctctccggatacgtgccagaatgcatgaagatcggaatcctcacacccatctataagaacaagggttctcgcaatgacgcatgcaactttcgcgggattaccgtgctccctgtcattgaaaagatcctggaaatggtcctgaaaattcggttggtccccaccctggagaaagatcaatctgcctttcagcgcggatttactgctaagacatctccacttcacgcagcattgatagtcgaagaggtatcacgagagtacaaggataaaggggaagatatagatttggttttcttggacgcaaaagctgcattcgatgtagtcgatcaccaccatctgctccgaaggctgtatcactctggcgtcaacgatcgccattggactatatttaaatcaatccacactcagtccacatcagtagtcaagtgggccaattcacgcctcgatccattcgaagttctgcagggagtcaggcaaggaggcatcacaagtacagacttgtataaaatatatataaacccactgcttaaacgacttgagatggctgaggagggatgcgtgatcggtgacgttagatgcaacacttccgcatgtgcagacgatgtaacacttaattctgagaagccaggagagacatccgtattgataagtatggcagagacatttgctaattacgaacggtacatacttcagcctaagaaaaccgaagccctgaaagtagtaaccagttcccgaaaggaaattgttgaagaggattttgaaatatacggcaataaaataaccaacaccgacacatgcacccatcttggtcttaaacggtcacacaccatatcttcaaccgccgaacagaacgttgataacaatatacaaaaagcccggcgcaccgtctacagtttcatgagttccggtttttacggagccggcggcttggatgtcccctctatactgcacatactcgaaatgcacattatccccattctcctttacggtctggaaattatacttcctaagaaaacccagattgacaaactggagatcttccaaaaacggattctgaagcagctcttggttcttccatctagtacccccgacattgctatatactcaatatccggacttctgcccgtgaaaattcaaatacacaagagagctttgatcatgtacaataacgtctgccttcaatccgagtccgctgttgagaggtgtatcgcagtcagacagctcaccgtgaaatccaataagagtgctagttggtttgttgatatacgcaagttgttctggctctatgaacttggggatccagaggatctgctggagtctcctaccgaaaaggaacactggaaacggacagtgaatcgcactattgacaacatctggctccaacaaactgtcgccgagtccaagactatgaagaccctccagaatttgaacatgaacatggttaaacctcgaaaaccccatccactgcttcagcagcaagccgtgagtacatacgacgcaaatagacaggtattgaagctgaaattcatgtgcggcaaatatattcttcagtcagatcgagcctctttctcaaagaacaaggttgatgatacatgtcgagtttgttataaaagtcctgaaaccctgcaacacatggtcttggagtgctccggtcttagtgctgtcagggatcctattttgcgagacatagagtcagaaatcgaaaaaagtttcccatgcctgtggcagtggtacacccaggatcagaaagttaccgcattgattgactgtactgtcttgtataaaaaacacaagctctcaaaggttgagtgccagagactccataagattgactttcagtgccgacgacttttctttaccctgcacactaagaggtttaagattttcgagctttcaaaaggaataaccgacactatgacattcgatttcgtaagggaacccggcaaaacatttaagcgcaagttgataggtttgtgttggaaagcctttcttgttacttactgtcgccgacatattttcccacttctcaccaaaagagtcaaaacagggctcgtaacagtaaagaaacacctgaaccaatacctggtgtccttgtccaaggaacggtaccatagtgtcaatcgaacaagggatctgcctgttgctaggcggaccccatacccactgcatcatggtgacttgatcaacagtagttctactgttccagaccatacttggcaattgctttcagctacacgaatactgtactagtaa

SEQ ID NO: 6 - DNA, 2127 bp, Macaca mulatta polyomavirus 1
atggataaagttttaaacagagaggaatctttgcagctaatggaccttctaggtcttgaaaggagtgcctgggggaatattcctctgatgagaaaggcatatttaaaaaaatgcaaggagtttcatcctgataaaggaggagatgaagaaaaaatgaagaaaatgaatactctgtacaagaaaatggaagatggagtaaaatatgctcatcaacctgactttggaggcttctgggatgcaactgagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatccattttctgttattgaggaaagtttgccaggtgggttaaaggagcatgattttaatccagaagaagcagaggaaactaaacaagtgtcctggaagcttgtaacagagtatgcaatggaaacaaaatgtgatgatgtgttgttattgcttgggatgtacttggaatttcagtacagttttgaaatgtgtttaaaatgtattaaaaaagaacagcccagccactataagtaccatgaaaagcattatgcaaatgctgctatatttgctgacagcaaaaaccaaaaaaccatatgccaacaggctgttgatactgttttagctaaaaagcgggttgatagcctacaattaactagagaacaaatgttaacaaacagatttaatgatcttttggataggatggatataatgtttggttctacaggctctgctgacatagaagaatggatggctggagttgcttggctacactgtttgttgcccaaaatggattcagtggtgtatgactttttaaaatgcatggtgtacaacattcctaaaaaaagatactggctgtttaaaggaccaattgatagtggtaaaactacattagcagctgctttgcttgaattatgtggggggaaagctttaaatgttaatttgcccttggacaggctgaactttgagctaggagtagctattgaccagtttttagtagtttttgaggatgtaaagggcactggaggggagtccagagatttgccttcaggtcagggaattaataacctggacaatttaagggattatttggatggcagtgttaaggtaaacttagaaaagaaacacctaaataaaagaactcaaatatttccccctggaatagtcaccatgaatgagtacagtgtgcctaaaacactgcaggccagatttgtaaaacaaatagattttaggcccaaagattatttaaagcattgcctggaacgcagtgagtttttgttagaaaagagaataattcaaagtggcattgctttgcttcttatgttaatttggtacagacctgtggctgagtttgctcaaagtattcagagcagaattgtggagtggaaagagagattggacaaagagtttagtttgtcagtgtatcaaaaaatgaagtttaatgtggctatgggaattggagttttagattggctaagaaacagtgatgatgatgatgaagacagccaggaaaatgctgataaaaatgaagatggtggggagaagaacatggaagactcagggcatgaaacaggcattgattcacagtcccaaggctcatttcaggcccctcagtcctcacagtctgttcatgatcataatcagccataccacatttgtagaggttttacttgctttaaaaaacctcccacacctccccctgaacctgaaacataa

SEQ ID NO: 7 - protein, 708 aa, Macaca mulatta polyomavirus 1
MDKVLNREESLQLMDLLGLERSAWGNIPLMRKAYLKKCKEFHPDKGGDEEKMKKMNTLYKKMEDGVKYAHQPDFGGFWDATEIPTYGTDEWEQWWNAFNEENLFCSEEMPSSDDEATADSQHSTPPKKKRKVEDPKDFPSELLSFLSHAVFSNRTLACFAIYTTKEKAALLYKKIMEKYSVTFISRHNSYNHNILFFLTPHRHRVSAINNYAQKLCTFSFLICKGVNKEYLMYSALTRDPFSVIEESLPGGLKEHDFNPEEAEETKQVSWKLVTEYAMETKCDDVLLLLGMYLEFQYSFEMCLKCIKKEQPSHYKYHEKHYANAAIFADSKNQKTICQQAVDTVLAKKRVDSLQLTREQMLTNRFNDLLDRMDIMFGSTGSADIEEWMAGVAWLHCLLPKMDSVVYDFLKCMVYNIPKKRYWLFKGPIDSGKTTLAAALLELCGGKALNVNLPLDRLNFELGVAIDQFLVVFEDVKGTGGESRDLPSGQGINNLDNLRDYLDGSVKVNLEKKHLNKRTQIFPPGIVTMNEYSVPKTLQARFVKQIDFRPKDYLKHCLERSEFLLEKRIIQSGIALLLMLIWYRPVAEFAQSIQSRIVEWKERLDKEFSLSVYQKMKFNVAMGIGVLDWLRNSDDDDEDSQENADKNEDGGEKNMEDSGHETGIDSQSQGSFQAPQSSQSVHDHNQPYHICRGFTCFKKPPTPPPEPET

SEQ ID NO: 8 - DNA, 2127 bp, Artificial sequence
  synthetic nucleotide sequence that encodes Macaca mulatta polyomavirus 1 large T antigen (TAG) (as shown in SEQ ID NO 7), but which has been codon optimized for expression by removal of codons that are not expressed well in dreissenid mussels
atggataaggtgctgaatagagaagaatccttgcaattgatggacctgcttgggcttgagaggtctgcttgggggaacattccacttatgcgaaaagcatacctcaaaaagtgtaaggagttccaccccgataagggcggtgacgaggagaagatgaagaaaatgaacactctgtacaaaaagatggaggacggggtaaagtacgcccatcaaccagattttggcggtttctgggacgctactgagattcctacctacgggaccgacgaatgggagcagtggtggaatgcttttaacgaagaaaacctcttttgttccgaggagatgccaagtagtgatgatgaggctacagcagacagtcagcacagtacccctccaaagaagaagagaaaagttgaagacccaaaggattttccaagtgagctgttgtctttcctctcccacgccgtcttctcaaaccgaacccttgcttgcttcgcaatatataccacaaaggagaaggcagctctgttgtacaaaaaaataatggagaaatacagtgtcaccttcataagtagacacaattcctataatcataatatactgttctttctcaccccacaccgccatagagtaagtgctataaataactacgcacagaagttgtgtaccttctccttcctgatttgcaaaggagtgaataaagagtacctgatgtactccgccttgacccgagatcccttctctgtaatcgaggaatccctccccggagggttgaaggagcatgattttaaccctgaagaggctgaggaaaccaagcaggtaagttggaaacttgttaccgaatatgcaatggagacaaaatgcgatgacgtattgcttctgttgggaatgtatctggaatttcagtactccttcgaaatgtgcctgaagtgtataaaaaaagagcagccctctcattacaagtaccacgagaaacactatgctaacgccgccatatttgcagattctaaaaaccaaaaaacaatttgtcaacaggccgttgataccgttctggcaaagaaacgggtcgactcattgcaactcaccagagagcagatgcttaccaaccgctttaacgatctgttggataggatggacattatgtttggctcaacaggatctgccgatattgaggaatggatggctggggtcgcatggctgcactgtctcctccccaaaatggacagtgtggtatatgatttcctcaagtgtatggtctataacatacccaaaaagcgctattggttgtttaaggggcccatagatagtggaaaaactacactcgctgccgcattgttggagctctgtggtggtaaagccttgaatgtcaacttgcccctggacagactcaactttgagttgggtgtcgctatcgaccaatttctcgtagttttcgaggatgttaagggcacaggcggggaatctagagacttgccatcaggacagggaatcaacaacctcgacaacctgagggattatctggacggctcagtaaaggtgaatttggagaaaaaacaccttaacaaacgcacacaaatcttccctcccggcatagtcaccatgaacgagtattccgtgcctaagaccctccaggctaggtttgtgaaacagattgatttcaggcctaaggattatctcaagcactgtctcgaacgatccgagttcctgctggagaaacggataattcagtctggaatcgcattgctgctcatgcttatctggtatcgacccgtagctgaatttgcacaaagtattcaaagtagaattgtggaatggaaggagaggcttgataaagagtttagtctttccgtctaccagaaaatgaaattcaatgtcgccatgggtataggtgtccttgattggctcagaaactcagacgacgacgacgaagactctcaggaaaatgcagataagaatgaggatggtggagagaagaacatggaggactctggccacgaaactggaattgattcccaaagtcaagggtcattccaagctcctcagtctagtcaatctgtacacgatcacaaccaaccttaccacatttgtcgggggtttacttgtttcaaaaagccaccaactccacctcccgagccagaaacatag

SEQ ID NO: 9 - protein, 88 aa, Dreissena bugensis
TNYPGDYGFEISFATPSKETKSTTWTYSETLKKLYVRMATTCPVRFKTARPAPQGAFIRTMPIFMKPEHVQDPVKRCPNHATSKEFNE

SEQ ID NO: 10 - protein, 88 aa, Mytilus galloprovincialis
TDYPGDYGFTISFSQPSKETKSTTWTYSESLKKLYVRMATTCPIRFKCLRQPPQGCVIRAMPIFMKPEHVQEPVKRCPNHATSKEHNE

SEQ ID NO: 11 - protein, 88 aa, Mya arenaria
TNYPGDYGFEISFATPSKETKSTTWTYSDILKKLYVRMATTCPVRFKTLRQPPPGCVIRSMPIFMKPEHVQEAVKRCPNHATSKEFNE

SEQ ID NO: 12 - protein, 88 aa, Spisula solidissima
TNYPGDYGFEISFATPSKETKSTTWTYSDMLKKLYVRMATTCPVRFKTNRQPPAGCIIRSMPIFMKPEHVQEAVKRCPNHATSKEFNE

SEQ ID NO: 13 - protein, 88 aa, Mytilus trossulus; identical to SEQ ID NO: 10

SEQ ID NO: 14 - protein, 88 aa, Mytilus edulis; identical to SEQ ID NO: 10

SEQ ID NO: 15 - protein, 88 aa, Crassostrea gigas
TDYAGDYGFQISFSQPSKETKSTTWTYSESLKKLYVRMATTCPVRFKSQRQPPAGCIIRAMPIFMKPEHVQEPVKRCPNHATSKENNE

SEQ ID NO: 16 - protein, 88 aa, Octopus bimaculoides
TNYPGDYHFEISFAQPSKETKSTTWTYSEKLDKLYVRMATTCPVRFKTLQTPPSGCQIRAMPIFMKPEHVQEVVKRCPNHATAKEHNE

SEQ ID NO: 17 - protein, 88 aa, Mizuhopecten yessoensis
TDYAGEHGFEISFSQPSKETKSTTWTYSEVLKKLYVRMATTCPVRFKCLRNPPPGCVIRAMPIFMKPEHVQETVKRCPNHATSKEHNE

SEQ ID NO: 18 - DNA, 23 bp, Artificial sequence
  Synthetic representative CRISPR/Cas9 guide nucleic acid sequence
actacacccacatcaacgctagg

SEQ ID NO: 19 - DNA, 23 bp, Artificial sequence
  Synthetic representative CRISPR/Cas9 guide nucleic acid sequence
acaactccctagcgttgatgtgg

SEQ ID NO: 20 - DNA, 23 bp, Artificial sequence
  Synthetic representative CRISPR/Cas9 guide nucleic acid sequence
tgtttgagagtccggggacatgg

SEQ ID NO: 21 - DNA, 23 bp, Artificial sequence
  Synthetic representative CRISPR/Cas9 guide nucleic acid sequence
tggcaaccacttgtccggtgagg

SEQ ID NO: 22 - DNA, 23 bp, Artificial sequence
  Synthetic representative CRISPR/Cas9 guide nucleic acid sequence
cttgaacctcaccggacaagtgg

SEQ ID NO: 23 - DNA, 23 bp, Artificial sequence
  Synthetic representative CRISPR/Cas9 guide nucleic acid sequence
cgggcggtcttgaacctcaccgg

SEQ ID NO: 24 - DNA, 23 bp, Artificial sequence
  Synthetic representative CRISPR/Cas9 guide nucleic acid sequence
gttctgatgaaggcgccctgggg

SEQ ID NO: 25 - DNA, 23 bp, Artificial sequence
  Synthetic representative CRISPR/Cas9 guide nucleic acid sequence
gcagcgcaccaggtgattggggg

SEQ ID NO: 26 - DNA, 23 bp, Artificial sequence
  Synthetic representative CRISPR/Cas9 guide nucleic acid sequence
gcacaagctggccaagtacgtgg

SEQ ID NO: 27 - DNA, 23 bp, Artificial sequence
  Synthetic representative CRISPR/Cas9 guide nucleic acid sequence
caagctggccaagtacgtggagg

(19) United States
(12) Patent Application Publication
(10) Pub. No.: US 2021/0392901 A1
(43) Pub. Date: Dec. 23, 2021

(54) DISSEMINATED NEOPLASIA CELLS AND METHODS OF THEIR USE TO CONTROL INVASIVE OR PEST SPECIES

(71) Applicant: BiomiLab, LLC, East Lansing, MI (US)

(72) Inventors: Steven T. Suhr, Okemos, MI (US); Marie-Claude Senut, Okemos, MI (US)

(73) Assignee: BiomiLab, LLC, East Lansing, MI (US)

(21) Appl. No.: 17/270,843

(22) PCT Filed: Aug. 29, 2019
(86) PCT No.: PCT/US2019/048912

§ 371 Date: Feb. 23, 2021

Related U.S. Application Data
(60) Provisional application No. 62/725,077, filed on Aug. 30, 2018.

(51) Int. Cl. A01N 63/10; C12N 15/90; C12N 5/07

(52) U.S. Cl. CPC A01N 63/10; C12N 2510/04; C12N 5/0601; C12N 15/90

(57) ABSTRACT

The current disclosure provides methods and compositions useful in preparing transformed and immortalized zebra and quagga mussel cells that function as disseminated neoplastic (DN) cells, as well as the cells produced thereby. Also provided are methods for using such mussel DNCs in cell culture, in vitro, and within live mussels in the lab or in the wild, to control mussel populations such as invasive zebra mussel or quagga mussel populations.

### CROSS-REFERENCE TO RELATED APPLICATION

This application claims priority to U.S. Provisional Application No. 62/725,077 filed on Aug. 30, 2018, which is incorporated herein by reference in its entirety as if fully set forth herein.

### STATEMENT REGARDING FEDERALLY SPONSORED RESEARCH OR DEVELOPMENT

This invention was made with government support under grant R19AC0002 awarded by the U.S. Bureau of Reclamation. The government has certain rights in the invention.

### FIELD OF THE DISCLOSURE

The current disclosure relates to methods and compositions for the control of invasive or undesirable species, particularly in a mixed population. It further relates to control of invasive mussel species using disseminated neoplasia cells.

### BACKGROUND OF THE DISCLOSURE

Disseminated neoplasia (DN) is lethal condition that can be used to suppress and kill invasive and pest species rapidly, efficiently, and with minimal or no potential for adverse effects on non-target species in the environment. DN is a type of cancer where the cancer cell itself is transmitted from one individual to another resulting in lethality. With the exception of fertilization, the transmission of living cells from one individual to another is quite rare, due primarily to the natural immune response in essentially all animals that rejects invading cells not recognized as “self”. Cells of DN develop with a loss of the cellular markers that distinguish cells from one individual from another within a species; however, they are still rejected by a host of another species. For instance, dog DN cells can successfully transfer from one dog to another resulting in lethal cancer, but these cells are rejected and harmless if introduced into humans or other non-dog species. For this reason, DN is a potent method of specifically suppressing and killing a specific invasive or pest species within a complex ecosystem or environment where a multitude of diverse species may be found.

DN is more common in marine organisms such as bivalves/mollusks because they lack complex immune systems that recognize foreign cells of the same species as non-self cells. Because mollusks—like mussels—live in an aqueous environment at high density and in large colonies, there is ample opportunity for cells of one individual to transfer over to a neighbor. Indeed, DN is a known “pathogen” of mussels that creates large die-offs in valuable blue mussel and Mediterranean mussel colonies that are farmed as a commercial food source.

Since DN is efficient at decimating mussel populations grown for food, it could also be used to control invasive pest mussel populations that threaten the waterways of the US, Canada, and many other countries. The primary species of mussels considered a threat to ecosystems are zebra (Dreissena polymorpha) and quagga (Dreissena bugensis) mussels, both very small mussels of the family Dreissenidae, whereas most other freshwater mussels native to America belong to other families such as Unionidae and Margaritiferidae. The molecular and cellular biology of different mussel species varies significantly, and the upshot of this is that DN that can flourish in one family of mussels is harmless to other types of mussels and mollusks (not to mention all other aquatic and terrestrial species).

### SUMMARY OF THE DISCLOSURE

Cells can disseminate and engraft between individual mussels within the same family or species. DN cells (DNCs) that will specifically suppress and kill a target invasive (mussel) species can therefore be created, for instance by selecting carcinogenic from among normal cells of an invasive species or by directly rendered cells carcinogenic by treatment with chemicals or manipulation of genes. In effect, laboratory produced DNCs such as those described herein are a pathogen specific to the family or species of mussel from which they are derived. These produced DNCs can be used in methods to control the corresponding target species, including among mixed populations and in the wild.

The current disclosure provides ways in which zebra/quagga mussel cells may be rendered transformed and immortalized into DN “cancer” cells, methods for how the DN cells are selected, expanded, and stored in cryogenic suspension, methods for how these DNCs are transmitted to live mussels in a controlled laboratory setting or in the wild, methods for how they may be refined and grown in cell culture in vitro or within live mussels either in the lab or in the wild, and methods for how they are monitored for dissemination and efficacy after deployment.

The strategy described herein emulates a natural process for the reduction of molluskan and mussel populations in the wild and provides an efficient, safe, and cost-effective solution to controlling invasive dreissenid mussels in the waterways of the United States and other affected countries.

As described herein, the target species (for instance, Genus Dreissena mussels such as zebra and quagga mussels) are obtained as a source of living cells. Live normal cells, such as mussel hemocytes (and other cell types), are harvested and cultured in vitro. DNCs are produced from hemocyte (and other cell type) cultures by one or more of: spontaneous generation of transformed cells, treatment of isolated cells with chemicals or agents that induce cellular transformation, genetic manipulation of isolated cells to induce the DNC phenotype using one or more of: knock out of p53 or other cell cycle regulating factor(s), increased expression of immortalizing protein(s) such as TERT, expression of known oncogene(s) such as SV40 Large-T antigen, or introduction into a single cell of multiple oncogenic factors.

Produced DNCs are isolated from normal cells and expanded as individual lines, for instance by expansion in vitro or by inoculation of live mussels for growth in vivo. DNCs may be concentrated and preserved indefinitely and for future use by cryogenic suspension and storage at −80° C. or in liquid nitrogen.

DNC lines are tested for efficacy (that is, the ability to infect and kill target organisms, such as target zebra or quagga mussels) by inoculation of live mussel cultures in a controlled laboratory environment and assayed for potency. Effective DNC lines can be selected and deployed on invasive zebra and quagga mussels in open water. This is done by one or more of: inoculation of mussels in the laboratory with DNCs followed by transplantation of infected mussels to targeted waterways where they infect the surrounding population, or direct introduction of DNCs to target mussel populations in open water. Optionally improved DNCs can be evolved and selected for by passage through host mussels.

Embodiments of the DNC provided herein are selective for infecting mussels of the same species from which the DNC was prepared, or selective for infecting mussels of the same Family as that from which the DNC was prepared. Though in some embodiments, such selective DNC will infect only members of the corresponding species (that is, for instance, quagga-derived DNC which infect only quagga mussels; or zebra mussel-derived DNC which infect only zebra mussels), or will only infect members of a Family (e.g., Dreissenidae mussels, rather than mussels from other Families) or a members of a Genus (e.g., Genus Dreissena mussels such as quagga and zebra mussels, rather than mussels form other Genera), in some examples “selective” does not require 100% species or Family exclusivity. Thus, in various embodiments a selective DNC will preferentially infect the corresponding species (or members of the same Family) by 100:1, a factor of 1000:1, or a factor of 10,000:1 or higher. Alternatively, a selective DNC will exhibit infection of non-self species (or non-self Family, or non-self Genus) at a rate of no more than 0.01%, no more than 0.001%, no more than 0.0001%, or not more than 0.00001% in a mixed population.

Thus, there is provided in a first embodiment an engineered disseminated neoplasia (DN) cell (DNC) from a Genus Dreissena mussel. In examples of this engineered DNC, the Genus Dreissena mussel is a quagga mussel or a zebra mussel. By way of example, the provided engineered DNCs in some examples includes one or more of: an immortalization mutation introduced using a carcinogenic agent; a knock out (deletion) mutation of p53 or another cell cycle regulating factor; a construct providing over expression of TERT or another immortalizing protein; or a construct providing expression of SV40 Large-T antigen (Tag).

Also provided is an engineered DNC, which is a quagga mussel DNC and which is capable of selectively infecting Genus Dreissena mussels in a mixed population. For instance, in examples of this embodiment, the engineered quagga mussel DNC is capable of selectively infecting quagga mussels in a mixed population.

Also provided is an engineered DNC, which is a zebra mussel DNC and which is capable of selectively infecting Genus Dreissena mussels in a mixed population. For instance, in examples of this embodiment, the engineered zebra mussel DNC is capable of selectively infecting zebra mussels in a mixed population.

Yet another embodiment provides an engineered disseminated neoplasia (DN) cell (DNC) from a Genus Dreissena mussel essentially as described herein. By way of example, such engineered DNC is from a quagga mussel or a zebra mussel.

Also provided are isolated disseminated neoplasia (DN) cells (DNCs) from a Genus Dreissena mussel essentially as described herein. Specific examples of this embodiment are isolated DNCs which are from a quagga mussel or a zebra mussel.

Another embodiment is an isolated immortalized Genus Dreissena mussel cell, such as for instance a quagga mussel cell or zebra mussel cell. In examples of the isolated immortalized mussel cell embodiment, the cell includes one or more of: a naturally occurring mutation giving rise to its immortalization; an immortalization mutation introduced using a carcinogenic agent; a knock out (deletion) mutation of p53 or another cell cycle regulating factor; a TERT over expression construct; or a SV40 Large-T antigen (Tag) expression construct.

Specific example isolated immortalized mussel cells are quagga mussel cells which are capable of selectively infecting Genus Dreissena mussels in a mixed population. In other examples, the isolated immortalized quagga mussel cell is capable of selectively infecting quagga mussels in a mixed population.

Additional specific example isolated immortalized mussel cells are zebra mussel cells which are capable of selectively infecting Genus Dreissena mussels in a mixed population. In other examples, the isolated immortalized zebra mussel cell is capable of selectively infecting zebra mussels in a mixed population.

Also provided are isolated immortalized Genus Dreissena mussel cells essentially as described herein, as well as isolated immortalized quagga mussel or zebra mussel cells essentially as described herein.

Yet another provided embodiment is a method of killing a Genus Dreissena mussel, which method includes infecting the mussel with an engineered DNC of any one of the herein provided embodiments, or with an isolated immortalized cell of any one of the herein provided embodiments. Examples of this method are a method of killing a quagga mussel and the engineered DNC is a quagga DNC or the isolated immortalized cell is a quagga mussel cell. Other examples of this method are a method of killing a zebra mussel and the engineered DNC is a zebra DNC or the isolated immortalized cell is a zebra mussel cell.

Also provided are methods of controlling a population of invasive, undesirable mussels including introducing to the population an engineered DNC as provided herein or an isolated immortalized cell as provided herein. In examples of this method, the invasive, undesirable mussels are Genus Dreissena mussels and the engineered DNC is a quagga mussel DNC or the isolated immortalized cell is a quagga mussel cell. For instance, in specific examples the invasive, undesirable mussels are quagga mussels and the engineered DNC is a quagga mussel DNC or the isolated immortalized cell is a quagga mussel cell. IN yet other examples of the method of controlling a population of invasive, undesirably mussels, the invasive, undesirable mussels are Genus Dreissena mussels and the engineered DNC is a zebra mussel DNC or the isolated immortalized cell is a zebra mussel cell. For instance, in specific examples of this embodiment the invasive, undesirable mussels are zebra mussels and the engineered DNC is a zebra DNC or the isolated immortalized cell is a zebra mussel cell.

In any of the described methods, the population of invasive, undesirable mussels is in some examples in a natural or constructed waterway or body of surface water. Thus, methods are provided for reducing invasive or pest mussel populations wherever such populations may be found, including in mixed ecological sites having other non-target mussel species as well as other non-mussel species.

Also provided is a method of producing an engineered disseminated neoplasia mussel cell or an isolated immortalized mussel cell essentially as described herein. In examples of this method, the mussel cell is a Genus Dreissena mussel cell, such as for instance a zebra mussel or quagga mussel cell.

Also provided is a method of killing a mussel cell essentially as described herein.

Yet another embodiment is a method of controlling a Genus Dreissena mussel population essentially as described herein. In examples of this embodiment, the mussel population includes quagga mussels, zebra mussels, or both.

### SEQUENCE LISTING

The nucleic acid and/or amino acid sequences described herein and provided in the accompanying Sequence Listing are shown using standard letter abbreviations, as defined in 37 C.F.R. § 1.822. Only one strand of each nucleic acid sequence is shown, but the complementary strand is understood as included in embodiments where it would be appropriate. A computer readable text file, entitled “25N4749.txt (Sequence Listing.txt)” created on or about Aug. 28, 2019, with a file size of 72 KB, contains the Sequence Listing for this application and is hereby incorporated by reference in its entirety.

SEQ ID NO: 1 shows the nucleotide sequence of D. bugensis (quagga mussel) p53.

SEQ ID NO: 2 shows the amino acid sequence of D. bugensis (quagga mussel) p53.

SEQ ID NO: 3 shows the nucleotide sequence of D. bugensis (quagga mussel) TERT.

SEQ ID NO: 4 shows the amino acid sequence of D. bugensis (quagga mussel) TERT.

SEQ ID NO: 5 shows a nucleotide sequence that encodes D. bugensis (quagga mussel) TERT (as shown in SEQ ID NO: 4), but which has been codon optimized for expression by removal of codons that are not expressed well in dreissenid mussels.

SEQ ID NO: 6 shows a nucleotide sequence that encodes Macaca mulatta polyomavirus 1 large T antigen (TAG), based on NCBI Reference Sequence: NC_001669.1 modified to remove intron sequence to produce the complete wild-type TAG open-reading-frame

SEQ ID NO: 7 shows the amino acid sequence of Macaca mulatta polyomavirus 1 large T antigen (TAG), GenBank #AAB59924.1

SEQ ID NO: 8 shows a nucleotide sequence that encodes Macaca mulatta polyomavirus 1 large T antigen (TAG) (as shown in SEQ ID NO: 7), but which has been codon optimized for expression by removal of codons that are not expressed well in dreissenid mussels.

SEQ ID NO: 9 shows the amino acid sequence encoded by Exon 6 of D. bugensis p53 (shown in FIG. 4).

SEQ ID NO: 10 shows the amino acid sequence of a portion of M. galloprovincialis p53 analogous to the amino acid sequence of encoded by D. bugensis Exon 6 (shown in FIG. 4); this sequence corresponds to GenBank AGK88244.1.

SEQ ID NO: 11 shows the amino acid sequence of a portion of M. arenaria p53 analogous to the amino acid sequence encoded by D. bugensis Exon 6 (shown in FIG. 4); this sequence corresponds to GenBank ACK28179.1.

SEQ ID NO: 12 shows the amino acid sequence of a portion of S. solidissima p53 analogous to the amino acid sequence encoded by D. bugensis Exon 6 (shown in FIG. 4); this sequence corresponds to GenBank AAQ55112.1.

SEQ ID NO: 13 shows the amino acid sequence of a portion of M. trossulus p53 analogous to the amino acid sequence encoded by D. bugensis Exon 6 (shown in FIG. 4); this sequence corresponds to GenBank AAT72302.1.

SEQ ID NO: 14 shows the amino acid sequence of a portion of M. edulis p53 analogous to the amino acid sequence encoded by D. bugensis Exon 6 (shown in FIG. 4); this sequence corresponds to GenBank AAT72301.1.

SEQ ID NO: 15 shows the amino acid sequence of a portion of C. gigas p53 analogous to the amino acid sequence encoded by D. bugensis Exon 6 (shown in FIG. 4); this sequence corresponds to GenBank CAJ85664.2.

SEQ ID NO: 16 shows the amino acid sequence of a portion of Octopus bimaculoides p53 analogous to the amino acid sequence encoded by D. bugensis Exon 6 (shown in FIG. 4); this sequence corresponds to GenBank XP_014784894.1.

SEQ ID NO: 17 shows the amino acid sequence of a portion of M. yessonsis p53 analogous to the amino acid sequence encoded by D. bugensis Exon 6 (shown in FIG. 4); this sequence corresponds to GenBank XP_021350070.1.

SEQ ID NOs: 18-27 show representative CRISPR/Cas9 guide nucleic acid sequences (shown in FIG. 5).

### BRIEF DESCRIPTION OF THE DRAWINGS

FIG. 1 shows a series of micrographs, which illustrate example of the use of Tag to immortalize target cells. In published experiments (Macpherson et al., J Cell Biochem. 91(4):821-39, 2004), cultured skeletal muscle cells were infected with a vector expressing the tsTag protein under the control of temperature and the drug doxycycline. The top panels show cells of three clonal lines of Tag-expressing cells proliferating unchecked at the permissive temperature of 33° C. and in the absence of tetracycline. The lower panels show that when these cells were shifted to 37° C. (the temperature that inactivates >90% of the tsTag molecules) and the expression of Tag was further suppressed by the addition of doxycycline, the skeletal muscle cells stopped proliferating and fused to one another, forming differentiated multinucleated myotubes. This experiment demonstrates that Tag expression pushes cells that would otherwise become non-proliferative and differentiated to continue dividing and display a cancer phenotype. In these images, the phase-contrast image of the cells has been overlayed by a fluorescent image revealing the nuclei stained with the fluorescent dye DAPI.

FIG. 2A-2C illustrate an example of genome modification using the CRISPR/Cas9 system. FIG. 2A is a schematic of a 300 bp PCR product flanking Exon 10 of the target gene. Four gRNA targets are spaced across the exon (T1-T4). A BgIII site was 140 bp from the 5′ end of the PCR product and lay directly on top of the T4 gRNA cut site. FIG. 2B illustrates a gel showing uncut PCR product from amplification of genomic DNA from cultured cells treated with Cas9 and each of the targeting gRNAs singly or in combinations as labeled. A single band of 300 bp represented either unmutated DNA or DNA mutated at a single site resulting in an indel of only a few bases that cannot be detected on the gel. The 270 bp band present in addition to the upper 300 bp band in some combination lanes, however, indicates cutting at two positions that then repair creating a large deletion. FIG. 2C illustrates a gel showing digestion with the enzyme BgIII revealed that in combinations containing T1+T4, almost no PCR product cuts with BgIII, indicating that both alleles in essentially all cells reflect either removal of sequence between T1 and T4 or are mutated within the T4 cut site alone. Subsequent analysis confirmed that T1+T4 cells have complete homozygous disruption of the target gene. A similar strategy will be used to provide genomic targeting in mussel cell DNA.

FIG. 3 is a schematic showing organization of the quagga mussel p53 gene upstream and around a critical p53 functional determinant peptide Arg-Cys-Pro-Asn-His (RCPNH) (positions 240 to 244 of SEQ ID NO: 2). Sequence analysis has determined the intron-exon structure of the quagga mussel p53 gene through coding exons 1-10; sequence information for the remainder of the gene past exon 10 has been collected but not yet analyzed to determine intron-exon boundaries. Quagga p53 coding exons 1-10 are similar in organization and size to intron-exon boundaries of p53 from other bivalve species, such as Mizuhopecten yessoensis (scallop) XM_021494392, Mytilus edulis AY705932.1, Mytilus trossus AY611471.1, and Mytilus galloprovincialis KC545827.1). Exon 6 of the quagga mussel p53 gene encodes the DNA binding domain including the critical RCXXH determinant critical for function.

FIG. 4 is an alignment of D. bugensis (Quagga mussel) Exon 6 amino acid sequence compared to the analogous region in related species. Identical or conserved amino acid substitutions are represented by a dot and non-conserved amino acids by a letter corresponding to the amino acid encoded. The RCXXH determinant (boxed) coordinates a zinc ion in the DNA binding pocket and is conserved among related mollusk species (shown in the figure), and essentially all known animal p53 proteins. The high level of conservation between the quagga p53 exon 6-encoded amino acids and the p53 of other species suggests that p53 of dreissenid mussels is structurally and functionally similar to all other p53s and predicts that mutations within or upstream of the quagga p53 RCXXH determinant will completely nullify protein function. The illustrated sequences are (in order): SEQ ID NOs: 9 to 17.

FIG. 5 illustrates sites at which mutations will be introduced into the quagga and zebra mussel p53 gene, upstream or proximal to the RCXXH determinant in exon 6, for instance using CRISPR/Cas9-induced mutation. CRISPR/Cas9 genomic targeting creates mutations by the introduction of insertions or deletions (“indels”) into the genomic sequence, resulting in a shift in the open reading frame (ORF) of encoded proteins. Indels will be introduced into the quagga (and zebra) mussel p53 genes by CRISPR/Cas9 targeting using any of the series of guide RNA (gRNA) target sequences shown in FIG. 5 (SEQ ID NOs: 18 to 27). Since loss of a functional RCXXH motif is sufficient to completely prevent p53 function, any mutation introduced upstream of this determinant in exon 6 will suffice to nullify p53. The location of seven high-efficiency gRNAs in the quagga mussel p53 gene that could produce mutations that would terminate p53 function are shown schematically and by sequence (SEQ ID NOs: 18 to 24) in FIG. 5. In addition, three lower-priority, exon 7 gRNA targets (SEQ ID NOs: 25 to 27) just downstream of the RCXXH determinant have been included, which may be employed as alternatives.

FIGS. 6A and 6B illustrate a system to over-express proteins such as TERT and SV40 Large T-Antigen (Tag) that can induce malignant transformation; this is an alternative to creating DN cancer cells by knock-out of p53 protein function. FIG. 6A shows a schematic of a representative plasmid vector that can be used for over-expression of the TERT, Tag, or other proteins to induce malignant transformation. Components of this vector include a strong ubiquitously-expressed promoter (i.e. ubiquitin or EF1a promoter), a 2A element that allows polycistronic expression, a selectable marker gene (i.e. for neomycin, puromycin, hygromycin, or zeocin-resistance), and a polyadenylation signal (i.e. signals from quagga mussel p53, TERT, or other genes). In addition to the use of an expression vector, the needed genetic components of the expression cassette described in FIG. 6A may be created using specific codons determined to promote efficient translation of genetic elements in dreissenid mussels (that is, codon-optimized for expression in dreissenid mussels). Codon optimization will overcome “codon bias” that can dramatically hinder protein production. The specific codons excluded from use in synthetic ORFs for use in dreissenid mussels are shown in FIG. 6B. In general, codons constituting less than 10-12% (0.1-0.12) of all codons used by a species are considered unfavorable and should be removed to increase protein production. Since expression cassettes may be more easily tested in mammalian cells than dreissenid tissues, mussel codon usage has been cross-referenced with mammalian codon usage to create a unique codon pool that excludes seven codons from use. As an example, a synthetic Tag ORF created herein incorporates the unique dreissenid/mammalian codon usage and other DNA sequence modifications that will facilitate use in mussels while preserving the Tag protein sequence and is shown in SEQ ID NO: 8. Similarly, a synthetic TERT ORF with optimized codon usage is provided in SEQ ID NO: 5.

### DETAILED DESCRIPTION

Like humans and most other animal species, marine bivalves can develop cancer (Carballal et al., J. Invertebr. Pathol., 131, 83-106, 2015). Malignant hemic neoplasia (HN)—analogous in some ways to leukemia in humans—is lethal to mollusks and has been studied extensively for its impact on species of commercial interest. Although HN was characterized as a pathological condition in mollusks several decades ago (Farley, 1969), it has only been revealed recently that some large-scale bivalve die-offs are caused by horizontal mollusk-to-mollusk direct transmission of HN cells (Carballal et al., J. Invertebr. Pathol., 131, 83-106, 2015, Metzger et al., Cell, 161, 255-263, 2015). Occurrences of horizontal transmission of cancer cells, or disseminated neoplasia (DN), are rare, but have been described, most notably in dogs (Murgia et al., Cell, 126, 477-487, 2006) and Tasmanian devils (Pearse & Swift, Nature, 439, 549, 2006). In molluskan populations, most research on this phenomenon has focused on understanding the environmental stressors and contaminants that lead to transformation of normal hemocytes to the cancerous phenotype. The objective of those studies was lessening or preventing DN lethality within threatened wild populations and commercially valuable stocks.

As described herein, the current disclosure turns this objective on its head and instead uses DN as a potent tool in the suppression and elimination of invasive mussel species. Cutting-edge methods of cell culture, genetic engineering, and genomic modification are applied to quagga and zebra mussels hemocytes to produce DN cells (DNCs) that will be used to transmit and foster lethal cancer specifically within these species. Using the strategy described herein, quagga and zebra mussels can be eliminated from infested waterways efficiently, economically, and with essentially no risk to other marine species, non-aquatic organisms, or humans.

Zebra and quagga mussels are obtained as a source of living cells. Live zebra and quagga mussels are obtained from captive cultures or from natural sources such as lakes and rivers.

Live normal mussel hemocytes are harvested and cultured. Hemocytes are roughly equivalent to mussel “blood”, but other cell types or a mix of hemocytes and other cells are included. For purposes of this disclosure, the term “hemocytes” is used to indicate both true hemocytes and all other cell types that are harvested from live mussels. These are extracted from quagga and zebra mussels as described in studies with mollusks (see, for instance, Elston et al., Dev. Comp. Immunol., 12, 719-727, 1988; Mateo et al., J. Fish Dis., 39, 913-927, 2016) and cultured using methods such as those suggested previously (see, for instance, Quinn et al., Cytotechnology, 59, 121-134, 2009; Kwoka et al., Mutation Research, 750, 86-91, 2013; Yoshino et al., Can. J. Zool., 91, 1-28, 2013). In various methods, the cells will be dispersed over 12 or 6-well plates and monitored over time cultured in a 12-18° C. incubator.

DNCs are produced (engineered) from hemocytes or other cell cultures by one or more of the following methods:

(A) Spontaneous generation and isolation of DNCs. By harvesting hemocytes and other cells from live zebra and quagga mussels and subjecting them to long-term continuous culture in vitro, spontaneously transformed cells of the DNC phenotype is generated and isolated as described below for use as the lethal DN reagent.

(B) Treatment with chemicals or agents that induce DNC transformation. Pools of wild-type hemocytes or other cells are treated with known carcinogenic agents to produce cells that exhibit uncontrolled growth and the neoplastic DNC phenotype. DNC cells are identified and harvested for use as the invention as described below.

(C) Genetic modification to induce DNCs by methods including:

[1] DNC creation by knock-out of the mussel p53 protein by targeted genomic disruption in cultured mussel cells (hemocytes or other cell types). CRISPR/Cas9 is one method by which targeted disruption is performed. Genomic disruption of target genes is performed by several methods including the widely popular CRISPR/Cas9 system (broadly described in Singh, 2015 and online at en.wikipedia.org/wiki/CRISPR). This methodology and others creates an insertion/deletion (indel) causing a frame-shift or a point mutation within a quagga or zebra mussel cell cycle control genes, such as the p53 (TP53) gene (Duffy et al., Europ. J. Cancer., 83, 258-265, 2017), resulting in complete loss of functional p53 protein within the cell. Hence, disruption of genes like p53 that halt cell division is sufficient to produce cell lines with uncontrolled, continuous growth that are the neoplastic cancer cells of this invention. Wild-type cultured mussel hemocytes are transfected with DNA and RNA and protein reagents using lipid carriers such as Lipofectamine® 2000, electroporation, or microinjection of linearized plasmid vector to introduce the mutational agents (i.e. CRISPR Cas9 reagents). Cells that display uncontrolled growth and the phenotype of disseminated neoplastic cells are isolated and expanded as individual cell lines for testing as functional DNCs as described below in Step 4.

[2] DNC creation by overexpression of the telomerase reverse transcriptase (TERT) protein by introduction of a plasmid or viral vector producing TERT from mussel species, scallop, or other species into cultured mussel cells (hemocytes or other cell types). Overexpression of the immortalizing and cancer-linked protein TERT (i.e. Choudhary et al., Front. Biosci. (Schol Ed)., 4, 16-30, 2012) will promote the neoplastic conversion of normal quagga and zebra mussel hemocytes or other cells. This method produces uncontrolled growth by the addition of new genetic material. An expression vector plasmid producing the TERT protein (or other immortalizing/transforming agent) is introduced into the normal mussel hemocytes using lipid carriers such as Lipofectamine® 2000 or by electroporation of linearized plasmid vector. Transformed mussel cancer cells are isolated and further processed as described below.

[3] DNC creation by expression of known oncogenes such as SV40 Large T-antigen (Tag) protein by introduction of a plasmid or viral vector producing the oncogene into cultured mussel cells. The introduction of Tag (see review of Tag action, see Ahuja et al., Oncogene, 24, 7729-7745, 2005) into normal mussel hemocytes or other cells will proceed essentially as described in Example 3b except that the Tag ORF is inserted into the transgene payload region of the vector. This plasmid is stably introduced into normal quagga and zebra mussel hemocytes or other cells, selected for neoplastic phenotype, and further processed as described below in Step 4.

[4] DNC creation by expression of a combination of oncogenic factors by introduction of plasmid or viral vectors producing the oncogenes into cultured mussel cells (hemocytes). If none of the individual factors of Methods [1-3] are sufficient on their own to induce neoplastic transformation, two or more different mutations, i.e. p53 knock-out+TERT over-expression, etc. will be combine to obtain DNCs. Other oncogenic proteins can also prove efficacious in combination with these methods.

Selection and quantification of DNCs. The production of DNCs from normal hemocytes, whether by targeted genomic mutation, the introduction of TERT, Tag, or other methods, is facilitated by the properties of neoplastic cells relative to their normal counterparts. First, DNCs have a distinct morphology compared to normal cells (Metzger et al., Cell, 161, 255-263, 2015). DNCs are rounded and appear very different from untransformed cells by light microscopy and can thus be easily identified and counted. Second, because they are non-adherent, they can also be readily separated away from untransformed cells that are stuck to the substrate. Third, while normal cells grow slowly and have a limited life, transformed cells will grow rapidly and are immortal. With continuous passage, it will be possible to “select” for cells that are transformed. These properties mean that regardless of the specific mutation introduced by any of the described methods (or equivalents thereof), all of the cells returned will by definition have mutations resulting in neoplasia. Even when the efficiency of targeting is only 0.1%, a handful of mutant cells is selectively expanded into a large DNC population.

Expansion of DNCs is performed long-term using DNCs grown by in vitro cell culture, grown in live-infected mussels maintained in the laboratory, or harvested from infected mussels in an open water environment.

Concentration and cryopreservation of DNCs. DNCs will be concentrated and cryopreserved. This allows for flexibility in their use in laboratory testing and facilitates their use in the field. DNCs will be concentrated by centrifugation and resuspended in freezing media that have as a base the medium used for growth of the cells combined with varying degrees of animal or fish serum, DMSO, glycerol, and other agents that prevent ice crystal formation. Aliquots of frozen cells will be stored in liquid nitrogen (LN₂) for later use.

Individual DNC lines are tested for efficacy by inoculation of live quagga and zebra mussels in a controlled laboratory environment and assayed for potency. DNCs are collected in their growth medium, pelleted by centrifugation, and resuspended at different concentrations for delivery to live mussels. Dosage of DNCs required for optimal inoculation will be empirically determined by measuring the rapidity of illness and death in target mussel cultures.

Once selected, DNC lines (for instance, the most potent line(s)) are deployed on invasive zebra and quagga mussels in open water. This is done by: 1) inoculation of zebra or quagga mussels with DNCs in the laboratory as described herein, followed by transplantation of infected mussels to targeted waterways where they infect the surrounding population, or 2) direct introduction of DNCs to target mussel populations in open water. DNC ampules will be maintained on dry ice until arrival at a high-density location of invasive mussels in the target waterway. Field scientists will thaw the frozen DNCs and inject a portion of them directly into the body of open mussels using a pipette. Alternatively, the DNCs will be placed in a heavier-than-water delivery substrate (i.e. glycerol) and deployed over target mussels as a cloud of cells. This process can be repeated at day/week intervals until active infection is detected. Mussel populations can be monitored for the development of disseminated neoplasia by sampling mussels or water in targeted areas and using histological methods, PCR, counting of live mussels, and/or other techniques to determine the need for additional deployment of DNCs

Evolution of improved DNCs by passage through host mussels. Serial inoculation in a laboratory setting can result in DNCs displaying superior properties of mussel-to-mussel transmission, more rapid growth and better survival. DNCs can also be evolved that are able to cross-inoculate both dreissenid species if they are not capable of doing so otherwise. This is accomplished by inoculating target mussels with a relatively large dose of cells introduced into the water, allowing early stage engraftment, and growth to a low level. DNCs would then be harvested and the process repeated 2-10 times. Cells with superior properties of engraftment will enter the animal earlier, grow faster, and increase as a percentage of the total DNC population each time the process is repeated.

Introduction: Invasive mussels pose a significant threat to US waterways such as the Great Lakes. There are also many challenges to targeting a marine species that is part of a complex ecosystem that is home to myriad other species, some physiologically and genetically similar to the target—that must be left as unaffected as possible by any ameliorative strategy. While chemical pesticides, pathogens, and mechanical/electrical barriers to invasive mussel infiltration and population growth may one day be developed, at present, “biological” barriers are the most cost-effective and efficient strategy available.

One of the most common types of biological barrier is the introduction of predator species to eliminate pest populations (i.e. Holmes et al., European Scientific Journal, May, 216-225, 2016). While this type of barrier works well in the home garden, the use of one novel species to combat another in a large and diverse environment like Lake Michigan carries many risks. Another highly effective type of biological barrier is one in which a subpopulation of the invasive species is captured or bred in captivity, rendered sterile, and then deployed in overwhelming numbers into the environment to “out-compete” their fertile, wild counterparts and thereby suppress reproduction. Probably the most famous and successful use of this approach has been the eradication of the screwworm fly, Cochliomyia hominivorax, by the US Department of Agriculture in North and Central America (Valter et al., Ionizing Radiations in Entomology, Evolution of Ionizing Radiation Research, Dr. Mitsuru Nenoi (Ed.), InTech, DOI: 10.5772/60409. Available online at: intechopen.com/books/evolution-of-ionizing-radiation-research/ionizing-radiations-in-entomology, 2015). A similar strategy is currently being tested by scientists in the State of Michigan and elsewhere in an attempt to control invasion of the great lakes and mid-west waterways by the sea lamprey, Petromyzon marinus (Great Lakes Fishery Commission: Sterile-Male-Release-Technique, http://www.glfc.org/pubs/FACT_6.pdf).

Another newly developed type of biological barrier that several groups have recently put forward as a strategy to combat invasive carp in US waterways, proposes the introduction of a genetic mutation that gradually eliminates the generation of females (Zhang, Transgenic disruption of aromatase using the daughterless construct to alter sex ratio in common carp, Cyprinus Carpio. A Master's Thesis, Auburn University, Aug. 6, 2016. Online at etd.auburn.edu/handle/10415/5325?show=full). This genetic alteration, referred to as the “daughterless mutation”, deletes the carp gene CYP19A1 encoding aromatase, an enzyme required for the conversion of androgen to estrogen and complete ovarian development in females. In the absence of aromatase, only functional males are produced that increasingly propagate the daughterless phenotype as they increase as a proportion of the overall population. In Danio rerio (zebrafish) carrying the daughterless mutation, complete abrogation of female fish from the population has been demonstrated (Lau et al., Sci. Rep., 6, 37357. PMID: 27876832, 2016).

Of the strategies outlined above, the seemingly best fit for invasive mussels might be the daughterless mutation strategy—but for four significant caveats. First, gene-based strategies require detailed knowledge of the genomic sequence of the targeted species and to date, comprehensive maps and sequences of the quagga and zebra mussel genome have not yet been completed or reported. Second, most gene-based strategies with a high probability of success will need to employ a gene drive—a type of genetic element that can “push” itself to homozygosity throughout a host population very quickly and thoroughly (Champer et al., Nat. Rev. Genet., 17, 146-159, 2016). The risk of a gene drive that renders a population unisex is that if it moves outside of its geographic target range, the species would be threatened in any new waterway, up to and including its original home range. In short, though unlikely, a gene-drive could inadvertently trigger world-wide extinction of the target species. The third caveat to using the aromatase-based daughterless strategy is that sex hormone regulation and sex determination may not work the same in mussels as in vertebrates such as carp and zebrafish and therefore may be ineffective. Finally, genome manipulation of mussel species by injection of fertilized zygotes has not yet been reported and may be problematic for purposes of creating modified strains. There are work-arounds that can minimize some of the caveats and limitations of strategies utilizing genomic modification; however, the ecological, methodological and technical hurdles remain daunting.

Disseminated neoplasia is a transmissible cancer lethal to mussels. With invasive mussels, there is another approach that is relatively unique to bivalves that could be employed to eliminate them rapidly, efficiently, and with essentially no potential for adverse effects on species native to US waterways. This unique approach uses a transmissible form of cancer known as disseminated neoplasia (DN), where cancer cells themselves are transmitted from one individual to another resulting in lethality (Carballal et al., J. Invertebr. Pathol., 131, 83-106, 2015). With the exception of fertilization, the transmission of living cells from one individual to another is quite rare, due primarily to the natural immune response in essentially all animals that rejects invading cells not recognized as “self”. The same immunity that protects a subject from infiltration by foreign species also blocks the transplantation of life-saving organs from within its own species without immunosuppressive intervention. Thus, just as a healthy kidney transplanted from one person to another cannot survive unaided in a foreign host body, cancer cells moved from one individual into another also cannot survive.

There are two well-known instances of disseminated cancer in mammals—canine transmissible venereal tumor (CTVT) and Tasmanian devil facial tumor disease (DFTD). CTVT (Murgia et al., Cell, 126, 477-487, 2006; Murchison, Oncogene, 27, S19-S30, 2008; Murchison et al., Science, 343, 437-440, 2014) is a DN in dog populations that was first described in by an English veterinarian in 1810, has spread across continents, and was recently genetically determined to have originated in a dog living more than 11,000 years ago (Murchison, Oncogene, 27, S19-S30, 2008; Murchison et al., Science, 343, 437-440, 2014). DFTD, first reported in 1996 and which has come extremely close to eliminating the wild Tasmanian devil populations in some habitats, has only recently been determined to also arise from the spread of live cancer cells from one devil to another through direct contact (reviewed in Bender et al., Annu. Rev. Anim. Biosci., 2, 165-187, 2014).

DN in mollusks was first described in the late 1960's and has since been studied extensively by marine biologists concerned for preservation of wild mollusks and mollusk populations with commercial importance (Carballal et al., J. Invertebr. Pathol., 131, 83-106, 2015). Although transmission can also be induced experimentally by injection of hemocytes from an infected animal to into uninfected animals using a syringe, in both the laboratory setting and in the wild it is clear that DN is transmitted from individual-to-individual by simple proximity. This mode of transfer has been experimentally reproduced by co-culture or healthy and cancerous mollusks within a shared tank (Elston et al., Dev. Comp. Immunol., 12, 719-727, 1988; Mateo et al., J. Fish Dis., 39, 913-927, 2016).

In the neoplastic cells of CTVT and DFTD, mutations have been identified that reduce their capacity to be recognized by the host immune system so that they can proliferate in new hosts. Proteins involved in self-recognition by the major histocompatibility complex (MHC) type I and II are suppressed, while the production of immunosuppressive cytokines is increased. Mollusks, on the other, lack an MHC system, and instances of both somatic and germ cell individual-to-individual transfer have been observed in some marine invertebrates, and “allografts” between proximal individuals may be natural and common in mollusks (discussed in Weiss & Fassati, Cell, 161, 191-192, 2015). Given that normal healthy cells are to some extent shared within mollusk populations, it is not surprising that neoplastic cells with unlimited growth potential rapidly travel from one mussel to another “infecting” the entire population.

Factors inducing neoplastic transformation. There are undoubtedly a number of mutations that can arise in mollusk (and mussel) hemocytes (and potentially other cell types) that can give rise to HN cells; however, it has been shown that one common perturbation of many molluskan DNs is alteration to the cell-cycle and cell death master regulating protein p53 (Walker et al., Adv. Mar. Biol., 59, 1-36, 2011; Diaz et al., Dis. Aquat. Organ., 90, 215-22, 2010; Vassilenko et al., Mutat. Res., 701, 145-152, 2010; Muttray et al., Comp. Biochem. Physiol. B. Biochem. Mol. Biol., 156, 298-308, 2010). p53 is the subject of thousands of studies for its role in cancer in many organisms, and mutations in p53 are widely considered to be the most common mutation in human cancers (Duffy et al., Europ. J. Cancer., 83, 258-265, 2017). Based on published reports linking changes in p53 to molluskan/mussel DN and the known role of p53 in neoplasia of mammals from mouse to man, it is predicted that mutation of the tumor suppressor p53 within the mussel genome also has a high probability of producing cancer, including HN.

Another key factor in the conversion of normal cells to cancer cells is over-expression of telomerase reverse transcriptase (TERT). TERT adds protective sequences known as telomeres to the ends of chromosomes to act as protective “bumpers” during the rigors of cell division. As cells divide, the telomeres are progressively eroded, eventually leading to direct damage to the chromosome, cellular dysfunction, and cell cycle arrest. In mammals, the progressive loss of telomeres results in the process describe as “aging”; however, it is this same process that prevents many cells in a body from growing uncontrollably and producing cancers (Pestana et al., J. Mol. Endocrinol., 58, R129-R146, 2017). In nearly all human and mammalian cancers, TERT, normally only expressed very early in development, is accidently turned on, permitting uncontrolled cell growth and tumor formation (i.e. Choudhary et al., Front. Biosci. (Schol Ed)., 4, 16-30, 2012).

TERT is a curious protein. One would imagine that all animals would express TERT in much the same way humans do; however, this is not the case. Some organisms, and aquatic organisms like teleost fishes such as zebrafish and carp, in particular, continue to express TERT for essentially their whole lives (Anchelin et al., Dis. Model Mech., 6, 1101-1112, 2013; Henriques et al., PLoS Genet., 9, e1003214. PMID:23349637, 2013; Carneiro et al., Dis. Model Mech., 9, 737-748, 2016). This helps to explain why koi (an ornamental strain of carp) kept healthy and well-fed in captivity in Japan have been recorded to live for more than two centuries (available online at fishlaboratory.com/fish/koi-hanako-longest-living-fish-ever). For these organisms, the rigors of their natural environment, predation, disease, and other factors are such strong determinants of longevity that robust health in old age—if it can be attained—is a better formula for survival of the species than a decreased risk of cancer due to TERT loss.

The expression pattern of TERT in mussels is thus far not reported in the scientific literature. If TERT in mussels is like it is in many fish, then sufficient TERT is likely present in mussel cells to support unlimited replication. If, on the other hand, TERT is expressed like it is in mouse (or man), then the addition of TERT to mussel hemocytes would be predicted to enhance their capacity to become neoplastic. In either event, it is likely that even if mussels express TERT at all stages of life, the addition of more TERT in mussel cells is likely to support the “immortalization” of cells and promote the neoplastic phenotype in general.

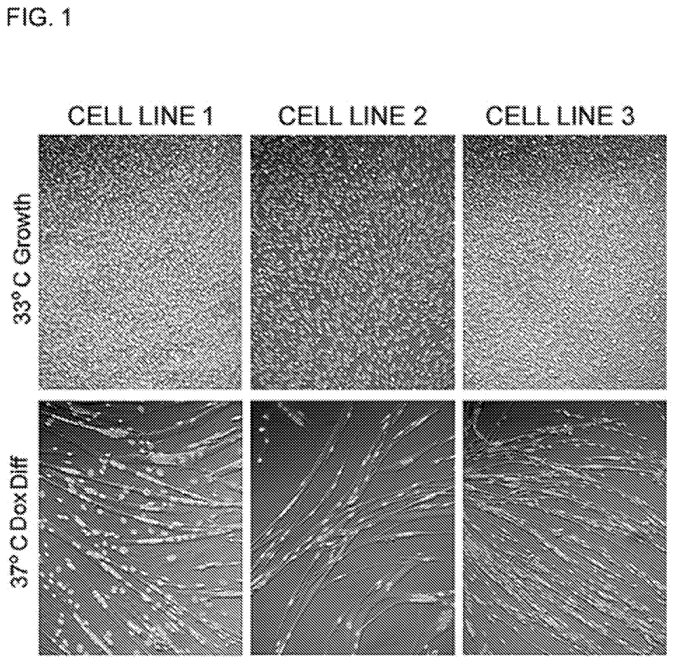

P53 and TERT are both endogenous factors that play central roles in the neoplastic transformation of cells; however, there are a number of exogenous factors—such as viruses—that produce extremely potent oncogenic agents. Scientists have used transforming factors derived from viruses to immortalize healthy normal cells and force them to divide and grow indefinitely. One such factor is the protein Large-T-Antigen (Tag) from Simian Vacuolating Virus 40 (SV40) (see review, Ahuja et al., Oncogene, 24, 7729-7745, 2005). The SV40 Tag protein has been shown to work through multiple cellular pathways to induce cellular transformation, most notably through inhibition of p53 and another tumor suppressive factor Rb. Temperature-sensitive forms of the SV40 Tag (tsTag) have been discovered that allow control over cellular immortalization by shifting cells containing the factor from a low temperature that induces transformation (usually 32° C.) to a non-permissive temperature that allows the cell to revert to normal growth and growth arrest (usually 37° C.). Scientists have used tsTAG to control growth and differentiation of skeletal muscle cells in vitro (FIG. 1). It is believed that Tag would have the same properties of cellular transformation in mussel cells that it has in mammalian, reptile, and amphibian cells.

Described herein are methods employing cutting-edge techniques of molecular and cellular biology (that is, genetic engineering techniques) to induce neoplasia in cultured quagga and zebra mussel cells (e.g., hemocytes), and to test these intentionally transformed cells for their ability to engraft to live quagga and zebra mussels, induce lethality, and disseminate throughout captive quagga and zebra mussel populations in a controlled laboratory environment. Seeding quagga and zebra mussels in the field with the genetically-modified DN cells (GMDNCs) to induce toxicity and spread throughout the invasive wild population in situ is also enabled. Ultimately, it is proposed that GMDNCs will eliminate invading quagga and zebra mussel populations within target waterways with no appreciable negative impact on the environment, native species, or the human population. Furthermore, it is expected that a biology-based suppression of this type is less likely to spread to home waters of quagga and zebra mussels than methodologies utilizing gene drive technology.

As used herein, the term “engineered” refers to a sequence (nucleic acid or amino acid), cell, or organism (e.g., mussel) that has been modified through intentional, laboratory action(s) so that it is no longer naturally occurring. Engineered sequences include, for instance, sequences with two or more portions that are not found together in nature (e.g., heterologous sequences that have been functionally fused together), as well as sequences that have been modified through intentional mutation (both random mutation that is intentionally induced, for instance through application of a mutagen; as well as specific genetic modifications, such as CRISPR/Cas9 modifications and other manipulations) and the polypeptides encoded by such mutated nucleic acid sequences. Engineered cells include, for instance, cells that have been intentionally modified to include (either in an autonomously replicating form or integrated into the genome of the cell) a heterologous sequence, or in which a native sequence has been intentionally mutated or modified. Engineered organisms include, for instance, organisms that contain a cell that has been intentionally modified to contains and/or express an engineered nucleic acid or polypeptide. “Genetic engineering” is a representative type of engineering. In general, an engineered modification is passed to progeny cells/organisms.

It is believed that cryopreserved GMDNCs will last for decades if not centuries in LN₂ storage, meaning that they could be re-deployed in the future at low cost should invasive dreissenid mussel re-infestation occur.

It is recognized that GMDNCs might cross inoculate non-dreissenid mussels or other mollusk species in target waterways. If this occurs, then application of this treatment technology in the field may imperil some wild indigenous species. This is a serious caveat, which might never be completely eliminated because it is impossible to assay GMDNCs against every possible freshwater mollusk let alone every other species in a wild environment. It is predicted that cross engraftment (beyond the Genus Dreissena, or the Family Dreissenidae) is unlikely for two reasons: 1) Data suggests that dreissenid mussels are physiologically quite different from other mussel species (further supported by data suggesting that quagga and zebra mussels have significantly different genomes compared to non-dreissenid mussels) and this would tend to inhibit GMDNC survival radically in non-self organisms (that is, organism other from a Family, or a Genus, or a Species, other than the Family/Genus/Species from which the source cells were obtained), and 2) although cross-species engraftment of HN has been observed in wild mollusks (Metzger et al., Nature, 534, 705-709, 2016), there are limited documented examples. It is expected that the herein described engineered HNCs and isolated immortalized mussel cells will be limited to engraftment only to dreissenid mussels and that if a low-level of engraftment can occur with other species, the resulting non-self infections are non-productive and cannot readily spread to other healthy individuals of the same species.

Quagga and zebra mussel genomes have only recently been described and are still in the early phases of characterization. Working with assistance from collaborators at the United States Bureau of Reclamation (USBR), the reagents and methods described herein have been developed.

Culture of mussels in the laboratory, extraction and culture of hemocytes and HN cells and transformation of cultured (other than mussel) cells using mutation of p53, TERT, and Tag have all been demonstrated in numerous studies to be effective. Even the mass-killing of mussel populations by DN in the field has been documented in wild mussel populations and is known to be rapid and efficient.

It is likely that a single concerted introduction of a treatment composition provided would be able to introduce a sufficient inoculant of GMDNCs into target waterways to produce a chronic infection that would disseminate throughout the invasive mussel population and cause population collapse. Furthermore, since GMDNCs cannot live indefinitely outside of a mussel host, once invading mussels are eliminated, GMDNCs are eliminated as well, leaving the environment free of any trace of the invasion or its cure.

Little if any potential for negative impact on other aquatic organisms, wild-life, or human populations is expected from the use of the technology described herein. GMDNCs are toxic only to mussels of the same species from which they are derived and cannot live in other host species. Even if some transfer to closely related species might occur, it is expected that such cross-species dissemination would be rare and non-productive. Furthermore, consumption of infected mussels or the GMDNCs themselves by other life-forms has no potential for deleterious effect. Even laboratory or field personnel exposed to high levels of GMDNCs during the production or infection process have no predicted health risk associated with use of these cells.

Because engineering and testing of the cells is performed in the controlled environment of the laboratory and a large number of GMDNCs can be produced and frozen for deployment when convenient, it is expected that the methods described herein will be cost effective.

It is believed that the transmission and fostering of an engineered form of mussel-specific lethal cancer will result in the total collapse of the quagga and zebra mussel populations in targeted waterways. In some embodiments, a single introduction of a sufficient inoculant of GMDNCs into target waterways will produce a chronic infection that will disseminate throughout the invasive mussel population.

Embodiments of the treatment are specific to invasive mussels without significant harm to non-target organisms, such as native mussels or threatened and endangered species. The technology described herein provides treatments that are toxic only to mussels of the same species from which they are derived; such treatment cells cannot live in other host species.

The described strategy specifically targets mussels and is not expected to significantly impact any other aspect of any ecosystem into which it is introduced.

It is believed that the treatments described herein are capable of application to large bodies of water, including for instance water bodies up to 160,000 surface acres and water volumes of 26,000,000 acre-feet. These treatments are amenable to use in waters with variable qualities and degrees of pollution. This treatment strategy is expected to have minimal or no negative impact on downstream water operations and facilities. GMDNCs are toxic only to mussels of the same species from which they are derived and cannot live in other host species. Furthermore, consumption of infected mussels or the GMDNCs themselves by other life-forms has no potential for deleterious effect. Treatment will therefore have minimal or no negative impact on water treatment or processing facilities and operations, as well as downstream water users. The strategy is not expected to impact recreational uses of waterways.

Representative specific sequences are provided herein, including the codon optimized sequences provided in SEQ ID NO: 5 (which encodes Dreissena bugensis (quagga mussel) TERT (as shown in SEQ ID NO: 4)) and SEQ ID NO: 8 (encodes Macaca mulatta polyomavirus 1 large T antigen (TAG) (as shown in SEQ ID NO: 7)). Also contemplated are functional variants of the provided specific nucleic acid and amino acid sequences. Such functional variants include nucleic acids (e.g., gene, pre-mRNA, mRNA) and polypeptides, polymorphic variants, alleles, mutants, and interspecies homologs that: (1) have an amino acid sequence that has at least 80%, 85%, 87%, 90%, 91%, 92%, 93%, 94%, 95%, 96%, 97%, 98% or 99% or greater amino acid sequence identity, preferably over a region of over a region of at least about 25, 50, 100, 200, 300, 400, or more amino acids, to a polypeptide encoded by a respectively referenced nucleic acid or an amino acid sequence; which variant maintains at least one biological function of the reference corresponding sequence.

The phrase conservatively modified variant(s) applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein or protein domain. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one type of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence with respect to the expression product (the polypeptide), but not with respect to specific, enumerated nucleic acid sequence(s). In general, however, the variants do not introduce, or tend to avoid introducing, into an encoding sequence codon(s) that are not well expressed in dreissenid mussels. That is, variant nucleic acids are generally codon optimized for repression dreissenid mussels.

As to amino acid sequences, one of skill will recognize that individual substitutions, deletions, or additions to a nucleic acid, peptide, polypeptide, or protein sequence which alters, adds or deletes a single amino acid or a small percentage of amino acids in the encoded sequence is a “conservatively modified variant”, where the alteration results in the substitution of an amino acid with a chemically similar amino acid. Conservative substitution tables that provide functionally similar amino acids are well known in the art. Such conservatively modified variants are in addition to and do not exclude polymorphic variants, interspecies homologs, and alleles of the invention. The following eight groups each contain amino acids that are considered conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

### EXEMPLARY EMBODIMENTS

1. An engineered disseminated neoplasia (DN) cell (DNC) from a Genus Dreissena mussel.
2. The engineered DNC of embodiment 1, wherein the Genus Dreissena mussel is a quagga mussel or a zebra mussel.
3. The engineered DNC of embodiment 1 or embodiment 2, which includes one or more of: a knock out (deletion) mutation of p53 or another cell cycle regulating factor; a construct providing expression of SV40 Large-T antigen (Tag); a construct providing over expression of TERT or another immortalizing protein; or an immortalization mutation introduced using a carcinogenic agent.
4. The engineered DNC of embodiment 3, in which: the knock out (deletion) mutation is generated using a CRISPR/Cas9 targeted mutation system; the expressed Tag is expressed from a nucleic acid sequence including the sequence of SEQ ID NO: 8; or the over expressed TERT protein is expressed from a nucleic acid sequence including the sequence of SEQ ID NO:5.
5. The engineered DNC of embodiment 4, in which: the knock out (deletion) mutation is in p53 and is generated using a CRISPR/Cas9 guide RNA (gRNA) target sequence selected from SEQ ID NOs: 18-27.
6. The engineered DNC of embodiment 1, which is a quagga mussel DNC and which is capable of selectively infecting Genus Dreissena mussels in a mixed population.
7. The engineered quagga mussel DNC of embodiment 6, which is capable of selectively infecting quagga mussels in a mixed population.
8. The engineered DNC of embodiment 1, which is a zebra mussel DNC and which is capable of selectively infecting Genus Dreissena mussels in a mixed population.
9. The engineered zebra mussel DNC of embodiment 8, which is capable of selectively infecting zebra mussels in a mixed population.
10. An engineered disseminated neoplasia (DN) cell (DNC) from a Genus Dreissena mussel essentially as described herein.
11. The engineered DNC of embodiment 10, which is from a quagga mussel or a zebra mussel.
12. An engineered disseminated neoplasia (DN) cell (DNC) from a Genus Dreissena mussel essentially as described herein.
13. The engineered DNC of embodiment 12, which is from a quagga mussel or a zebra mussel.
14. An isolated immortalized Genus Dreissena mussel cell.
15. The isolated immortalized mussel cell of embodiment 14, which is a quagga mussel cell or zebra mussel cell.
16. The isolated immortalized mussel cell of embodiment 14 or embodiment 15, which includes one or more of: a knock out (deletion) mutation of p53 or another cell cycle regulating factor; a SV40 Large-T antigen (Tag) expression construct; a TERT over expression construct; a naturally occurring mutation giving rise to its immortalization; or an immortalization mutation introduced using a carcinogenic agent.
17. The isolated immortalized mussel cell of embodiment 16, in which: the knock out (deletion) mutation is generated using a CRISPR mutation system; the expressed Tag is expressed from a nucleic acid sequence including the sequence of SEQ ID NO: 8; or the over expressed TERT protein is expressed from a nucleic acid sequence including the sequence of SEQ ID NO: 5.
18. The isolated immortalized mussel cell of embodiment 17, in which: the knock out (deletion) mutation is generated using a CRISPR/Cas9 guide RNA (gRNA) target sequence selected from SEQ ID NOs: 18-27.

19. The isolated immortalized mussel cell of embodiment 15, which is a quagga mussel cell and which is capable of selectively infecting Genus Dreissena mussels in a mixed population.

20. The isolated immortalized quagga mussel cell of embodiment 19, which is capable of selectively infecting quagga mussels in a mixed population.
21. The isolated immortalized mussel cell of embodiment 15, which is a zebra mussel cell and which is capable of selectively infecting Genus Dreissena mussels in a mixed population.
22. The isolated immortalized zebra mussel cell of embodiment 21, which is capable of selectively infecting zebra mussels in a mixed population.
23. An isolated immortalized Genus Dreissena mussel cell essentially as described herein.
24. An isolated immortalized quagga mussel or zebra mussel cell essentially as described herein.
25. A method of killing a Genus Dreissena mussel, including infecting the mussel with an engineered DNC of any one of embodiments 1-13 or an isolated immortalized cell of any one of embodiments 14-24.
26. The method of embodiment 25, which is a method of killing a quagga mussel and the engineered DNC is a quagga DNC or the isolated immortalized cell is a quagga mussel cell.
27. The method of embodiment 25, which is a method of killing a zebra mussel and the engineered DNC is a zebra DNC or the isolated immortalized cell is a zebra mussel cell.
28. A method of controlling a population of invasive, undesirable mussels including introducing to the population an engineered DNC of any one of embodiments 1-13 or an isolated immortalized cell of any one of embodiments 14-24.
29. The method of embodiment 28, wherein the invasive, undesirable mussels are Genus Dreissena mussels and the engineered DNC is a quagga mussel DNC or the isolated immortalized cell is a quagga mussel cell.
30. The method of embodiment 29, wherein the invasive, undesirable mussels are quagga mussels and the engineered DNC is a quagga mussel DNC or the isolated immortalized cell is a quagga mussel cell.
31. The method of embodiment 28, wherein the invasive, undesirable mussels are Genus Dreissena mussels and the engineered DNC is a zebra mussel DNC or the isolated immortalized cell is a zebra mussel cell.
32. The method of embodiment 31, wherein the invasive, undesirable mussels are zebra mussels and the engineered DNC is a zebra DNC or the isolated immortalized cell is a zebra mussel cell.
33. The method of any one of embodiments 28-32, wherein the population of invasive, undesirable mussels is in a natural or constructed waterway or body of surface water.
34. A method of producing an engineered disseminated neoplasia mussel cell or an isolated immortalized mussel cell essentially as described herein.
35. The method of embodiment 34, wherein the mussel cell is a Genus Dreissena mussel cell.
36. A method of killing a mussel cell essentially as described herein.
37. A method of controlling a Genus Dreissena mussel population essentially as described herein.
38. The method of embodiment 37, wherein the mussel population includes quagga mussels, zebra mussels, or both.

### EXAMPLES

### Example 1. Harvest and Culture of Dreissenid Mussel Hemocytes

Example 1.a. Establishment of live colonies. The first step of Example 1 is to establish small colonies of live quagga, zebra, and unionid (or other control) mussels within a secure facility. Live mussels will be collected, for instance with the help of State Department of Natural Resources personnel and in accordance with permit(s) to collect and culture the mussel species.

Mussels will be cultured in multiple aquaria with ambient temperature control and the use of tank heaters/coolers to vary temperatures to the preferences of each species. A light-dark cycle produced by natural daylight will be maintained. Mussels will be inspected, fed, and their tanks cleaned at intervals to ensure healthy animals. In general, conditions for the establishment and support of mussel cultures will be as described in references such as Elston et al. (Dev. Comp. Immunol., 12, 719-727, 1988).

Example 1.2. Harvest and culture of live normal hemocytes and other cell types. Hemocytes or other cell types will be extracted from quagga and zebra mussels as described in similar studies with mollusks (i.e. Elston et al., Dev. Comp. Immunol., 12, 719-727, 1988; Mateo et al., J. Fish Dis., 39, 913-927, 2016) and cultured using methods suggested by several publications (i.e. Quinn et al., Cytotechnology, 59, 121-134, 2009; Kwoka et al., Mutation Research, 750, 86-91, 2013; Yoshino et al., Can. J. Zool., 91, 1-28, 2013). For hemocytes, a needle and syringe will be inserted into the adductor muscle of the live mussel and fluid withdrawn containing 100-150 μl of cells. Extracted cells will be pooled and centrifuged at low speed (1100 rpm) to pellet cells. The pelleted hemocytes will be resuspended in sterile mussel cell medium (MCM). As devised by Quinn et al. (Cytotechnology, 59, 121-134, 2009), MCM is “15% Leibovitz L-15 media consisting of (1 L): 150 mL Leibovitz L-15 (Gibco), 5 mL Penicillin-Streptomycin (5,000 IU/mL-5,000 μg/mL, Gibco), 2 mL Gentamicin (50 mg/mL, Gibco), 0.01 g Kanamycin (759 μg/mL, Sigma), 0.01 g Phenol red (Sigma), 843 mL Sterile water (Sigma), and 2.38 g HEPES (Gibco)”. MCM osmolarity and pH are regulated to 80-100 mOSM and 7.5 respectively, and the medium is sterile filtered and stored for up to 6 months at −20° C. Cell types other than hemocytes may be produced by microdissection of individual tissues, dissociation by mechanical or enzymatic digestion, and dispersion in plates and culture as described above and below.

The cells will be dispersed over 12 or 6-well plates and monitored over several days of culture in a 15-18° C. incubator. Trypan blue exclusion will be used to examine the number of live cells in culture at time intervals and cells will be stained with the fluorescent stain Hoechst 33342 and imaged on a fluorescent microscope to examine cell and nuclear morphologies. It is expected that this will result in reproducible extraction of live hemocytes from individual mussels, reproducible culturing of such cells, with predictable numbers and aspects of the surviving cells.

### Example 2. Conversion of Dreissenid Mussel Hemocytes to Genetically-Modified Hemic Disseminated Neoplasia Cells (GMDNCs) and Comparison to Normal Hemocytes

This example describes representative methods for long-term culture, expansion, and cryopreservation of GMDNCs.

Example 2.1. Production of transforming agents for immortalization of dreissenid mussel hemocytes. There are several good candidate genes as targets for promoting neoplastic transformation of mussel hemocytes. The following will be tested:

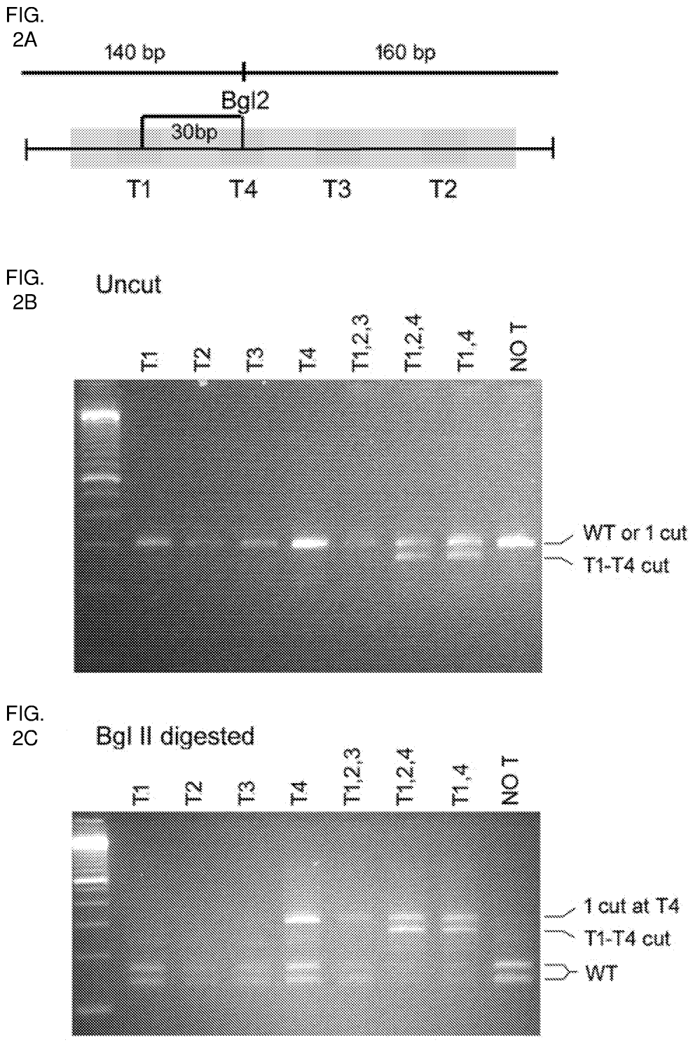

Example 2.1a Targeted disruption of the quagga and zebra mussel p53 gene by CRISPR/Cas9. Significant success has been accomplished with genomic disruption of target genes using the widely popular CRISPR/Cas9 system (broadly described in Singh, 2015 and available online at en.wikipedia.org/wiki/CRISPR). An example of targeted genomic mutation using the CRISPR/Cas9 system on cultured mammalian cells is shown in FIG. 2. As shown, with high-quality gRNA target sequences used singly or in groups, mutations can be introduced into both alleles of a gene within large cell populations (20K cells were targeted in the experiments of FIG. 2) resulting in complete knock-out of function. This same methodology will be employed to create an insertion/deletion (indel) causing a frame-shift or a point mutation within the critical DNA binding domain of the quagga and zebra mussel p53 gene, resulting in complete loss of functional p53 protein within the cell.

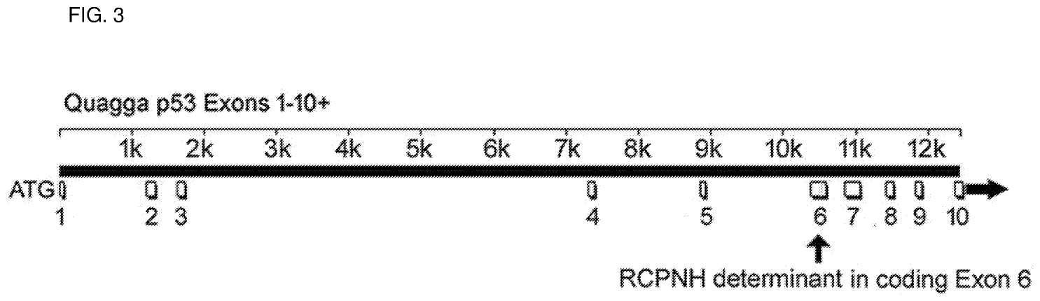

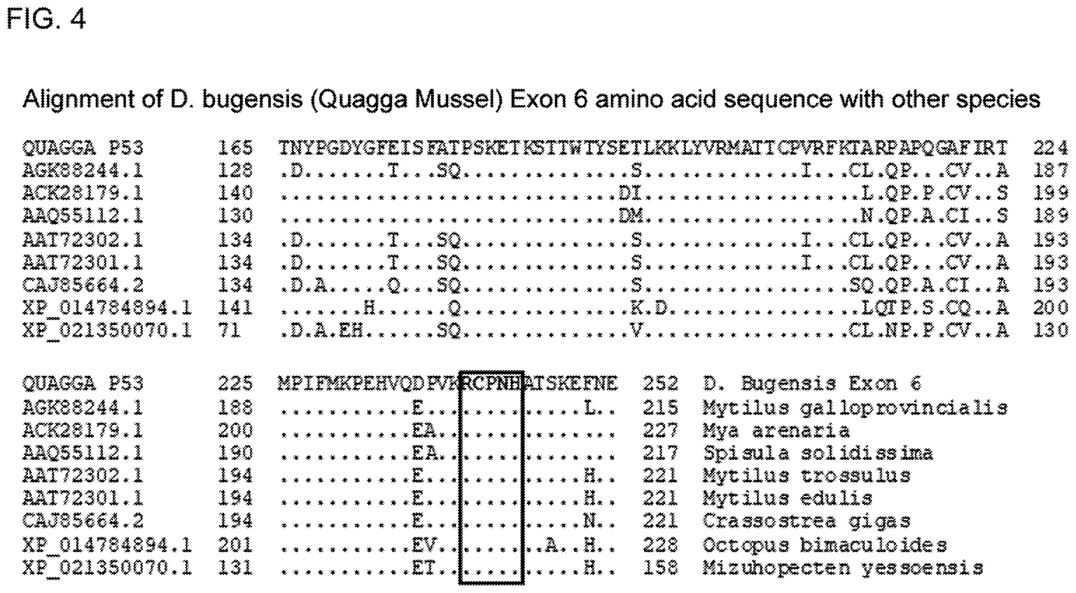

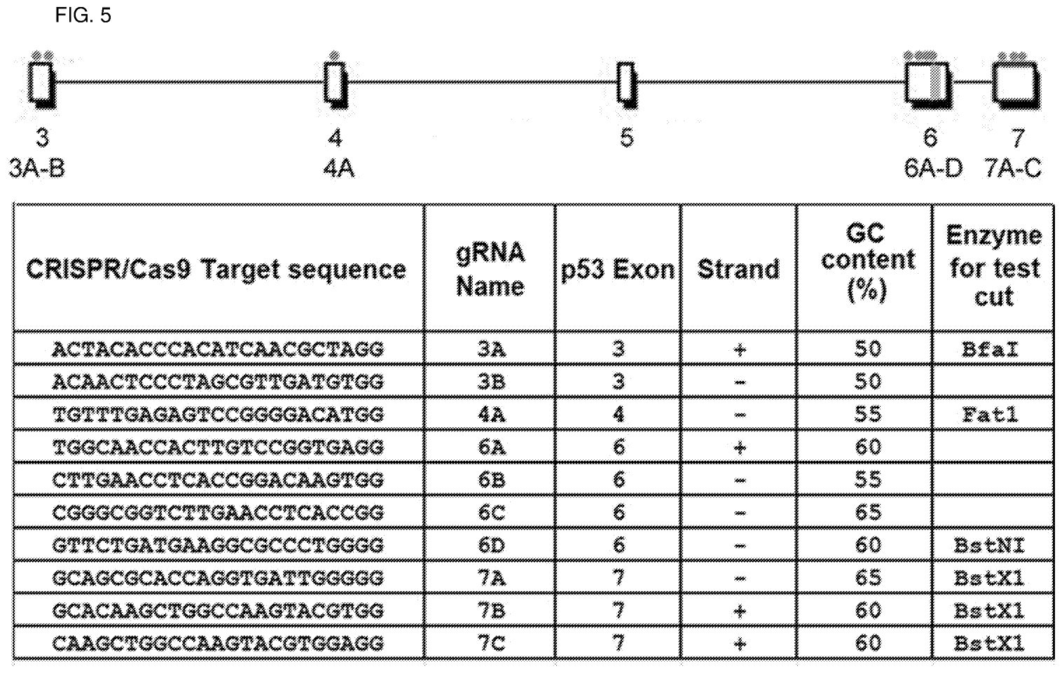

To this end, the structure of the p53 gene has been determined using data from the quagga mussel genome (provided by collaborators at the USBR) (FIG. 3). The overall pattern of exons and introns are similar to organization of the p53 genes of other species, and exon 6 is of particular interest because it is highly conserved across species (FIG. 4) and because it encodes the protein motif most frequently mutated in p53 in cancers. This motif, RCXXH (FIG. 4, boxed area below asterisks) is a critical portion of the protein interacting with zinc ions to form the DNA binding pocket, and mutation of the R, C, or H residues essentially destroys p53 functionality (Blanden et al., Drug Discov. Today, 20, 1391-1397 2015). gRNAs targeting the DNA sequence proximal to the RCXXH motif, even if they do not produce an indel causing a catastrophic frame-shift mutation, would likely impact zinc binding and therefore p53 function. FIG. 5 shows 10 Cas9 gRNA targets proximal to the RCXXH region in the M. gallo p53 gene. Seven of these targets (indicated on the schematic as gray dots) are located upstream of the RCXXH motif in Exon 6 (indicated by gray shading) and three additional targets are downstream in exon 7. Cutting of genomic DNA at any of these 10 targeted sites introducing a frame-shift mutation would be predicted to completely nullify p53 protein activity. The gRNA sequences targeting each of the 10 high-efficiency targets are shown in FIG. 5B. Disruption of several of these targets would result in the mutation of the restriction endonuclease sites shown in the last column of the table, and these enzymes will be used to determine the efficiency of targeting mussel p53 in a manner analogous to the data shown in FIG. 2.

At least three of the 10 gRNAs (or 30%) in FIG. 5B should be 80-95% effective at cutting and mutating their genomic target. The 10 candidate gRNAs will be synthesized as short RNA molecules that will be complexed with a tracer RNA to form the RNA-guided component of the endonuclease. The RNA components will be mixed with pure 3-NLS-Cas9 protein (Alt-R system from IDT—available online at idtdna.com/pages/docs/default-source/CRISPR/alt-r-crispr-cas9-system-user-guide.pdf.) and then transfected into recipient cells in vitro using lipid-based transduction reagents, electroporation, or direct microinjection.

To determine the conditions best suited to transduction of mussel hemocytes with CRISPR RNA components, fluorescent reporter vectors or RNAs encoding eGFP, eYFP, dsRED, or other fluorescent reporters will first be used on target cells. By measuring the intensity of fluorescence at different time intervals post-transduction, conditions will be identified that are likely to be effective with the Alt-R components. This same strategy has been used with a multitude of cell types from other species. 24-48 hours post-transfection, the medium of target cells will be changed and the cells passaged to promote recovery from the procedure.

After cells have recovered and expanded, a portion of the cells will be harvested and DNA extracted. PCR will be performed on the DNA using primers flanking the target region (i.e. in FIG. 2 the flanking primers generate a 300 bp PCR product) and then the PCR product will be assayed for changes to the DNA by T7 endonuclease digestion, restriction endonuclease digestion (as shown in the last column of the table in FIG. 5B), or cloning and sequencing. Assays of these types have been performed on many occasions, and it is believed that these methods will enable detection and determination of the efficiency of indel formation within the quagga and zebra mussel p53 exon targets. The cells not harvested to make DNA will be further cultured and monitored for signs of neoplasia.

Example 2.1b Transformation/Immortalization of quagga and zebra mussel hemocytes by overexpression of TERT. As indicated in the introduction, overexpression of the TERT protein is predicted to promote the neoplastic conversion of normal quagga and zebra mussel hemocytes. Unlike Example 2.1.a where endogenous DNA sequence is “subtracted” to produce the loss of function of a gene that keeps uncontrolled cell growth in check, this sub-example seeks to promote uncontrolled growth by the addition of new genetic material.

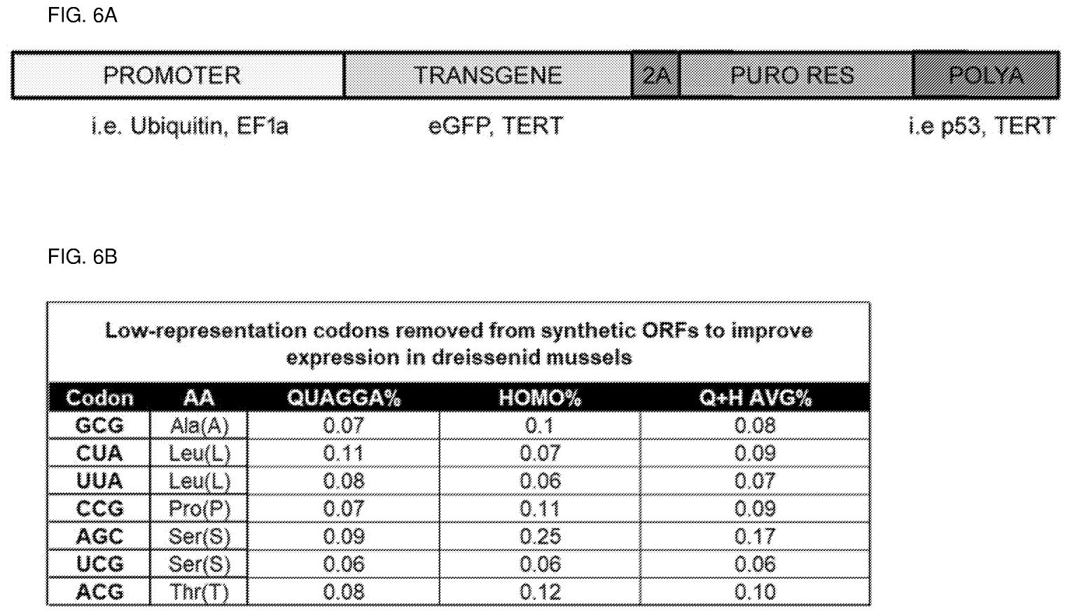

Described herein is a synthetic quagga mussel TERT ORF (SEQ ID NO: 5) that encodes the native quagga TERT protein sequence (SEQ ID NO: 4) but using the unique codon pool described above and in FIG. 6B. This synthetic TERT ORF will be used in the production of a mussel TERT over-expression vector using the components shown in FIG. 6A and described in greater detail below.

Expression vectors for use to transform mussel cells, like all expression vectors, will have several components. The following are specific examples of components that can be used in such a vector. First, it will require a plasmid backbone in which to assemble the multi-part expression vector. To this end, the common shuttle vector pUC19 can be used with ampicillin resistance in XL-blue (K-12-derived) attenuated E. coli. Second, a promoter for high-level expression of the TERT (or other) ORF will need to be included. Some promoters are known to function at high levels in other marine organisms (primarily zebrafish and xenopus). These include the ubiquitin promoter, the EF1a promoter, or the medaka beta-actin promoter, which are known to function efficiently across multiple species (Mosimann et al., Development, 138, 169-177, 2011, Yoshinari et al., Dev. Growth Differ., 54, 818-828, 2012). The third component is the TERT ORF (such as SEQ ID NO: 5). However, to validate the expression system, an ORF for a fluorescent reporter protein such as eGFP will first be used to examine transduction efficiency, promoter expression, and other parameters. Following TERT or eGFP, a cassette encoding a 2A element and an ORF encoding resistance to the antibiotic puromycin (Puro) can be added. The 2A element allows co-production of two proteins in tandem simultaneously—for instance, TERT/eGFP upstream of the 2A element and the puro-resistance ORF in the downstream position. By expressing the resistance gene along with the transgene, cells expressing eGFP or TERT can be isolated by selection of cells with the puromycin drug added to the growth medium. Other antibiotic resistance genes could be alternatively used. This strategy has been used many times to stably express a variety of proteins in transduced cultured cells.

The final component of the expression cassette will be a polyadenylation sequence needed to promote polyadenylation of the mRNA encoding the expressed proteins. By way of example, the sequence derived from the 3′ end of mussel genes such as p53 or TERT itself that contain the polyA signal consensus, can be used to promote polyA tailing of the transcript.

Each of these components will be assembled from synthetic DNAs or DNAs produced by PCR amplification or equivalent in a step-wise fashion in the pUC backbone. Variant plasmids encoding the eGFP transgene will be introduced into normal mussel hemocytes using lipid carriers such as Lipofectamine® 2000, electroporation, or microinjection of linearized plasmid vector. The efficiency of different transduction methods will be compared and modified over several rounds to maximize the number of cells transduced and expressing the fluorescent reporter or resistant to the antibiotic puromycin. Once the method and vector composition giving the best results are identified, the TERT-encoding plasmid will be utilized and cells placed under puromycin selection to eliminate un-transduced cells. Cells will be monitored at intervals for changes in morphology and growth consistent with neoplastic transformation. Those cultures giving rise to HN cells will be continued and further expanded as described below.

Example 2.1c Transformation/Immortalization of quagga and zebra mussel hemocytes by overexpression of Large T-antigen (Tag). The introduction of Tag into normal mussel hemocytes or other cells will proceed almost identically to procedures described in Example 2.1b except instead of the TERT ORF, the Tag ORF will be inserted into the best expression vector identified above. The temperature-sensitive Tag variant has been used in earlier experiments; it may optionally continue to be used in mussel experiments even though temperatures for growth of live mussels or mussel cells will Generally be below the temperature threshold required for inactivation of Tag (>36° C.). Even though Tag will never be thermally inactivated in mussel cells, there is increased safety for personnel working with the vectors in case of inadvertent introduction of the vector since normal human body temperature is sufficient to render the tsTag non-functional. The synthetic Tag ORF (SEQ ID NO: 8, for instance) will utilize the same restricted codon pool described in FIG. 6B and may include several silent restriction sites within the sequence to facilitate conversion of the wild-type TAG protein encoded to temperature-sensitive forms by replacement of Alanine 438 by Valine and/or replacement of Arginine 357 to Lysine (numbered as in SEQ ID NO: 7). This special Tag ORF will be inserted into the transgene payload region of the vector described in FIG. 6A for introduction into normal quagga and zebra mussel cells, selected, and processed as described with plasmids in Example 2.1b above.

Example 2.1d. Combining multiple oncogenic factors. If none of the individual factors of Examples 2.1a-2.1c are sufficient on their own to induce neoplastic transformation, the different mutations, i.e. p53 knock-out+TERT over-expression, etc. can be combined to obtain GMDNCs. Additional oncogenic proteins may also be tested, for instance using the same expression vector, if none of the factors described above are successful.

Selection and quantification of neoplastic cells. The production of HN cells from normal hemocytes, whether by targeted genomic mutation, the introduction of TERT, Tag, or other methods, is facilitated by the properties of neoplastic cells relative to their normal counterparts. First, HN cells have a distinct morphology compared to normal cells. As shown in FIG. 1B, 1C of Metzger et al. (Cell, 161, 255-263, 2015), HN cells are rounded and appear very different from untransformed cells by light microscopy and can thus be easily identified and counted. Second, because they are non-adherent, they can also be readily separated away from untransformed cells that are stuck to the substrate. Third, while normal cells grow slowly and have a limited life, transformed cells will grow rapidly and are immortal. With continuous passage, cells that are transformed can be “selected” for. These properties mean that regardless of the specific mutation introduced by CRISPR/Cas9 targeting, all of the cells returned will by definition have mutations resulting in neoplasia. Even if the efficiency of targeting is only 0.1%, a handful of mutant cells are predicted to be able to be expanded into a large HN population.

Example 2.2. Optimizing concentration and cryopreservation of GMDNCs. Ultimately, use of GMDNCs will be simplified if they can be concentrated and cryopreserved. This would allow flexibility in their characterization and would also contribute to their eventual use in the field. To this end, GMDNCs will be concentrated by centrifugation and resuspended in different freezing media used commonly in the cryopreservation of cells from other species. As a starting point for preservation methods, the report by Kwok et al. (Mutation Research, 750, 86-91, 2013) can be used. Most of these media have as a base the medium used for growth of the cells combined with varying degrees of animal or fish serum, DMSO, glycerol, and other agents that prevent ice crystal formation. 5-6 media and 3-4 different freezing regimens (rate of cooling, concentration of cells, etc.) will be devised to identify the best method. Aliquots of frozen cells will be stored in liquid nitrogen (LN₂), and thawed at intervals to assay and compare survival. Methods with the best results will be further varied in an effort to maximize efficiency.

The work in Example 2 will result in cultured HNCs produced by at least two methods for use in live mussels in Example 3.

### Example 3. Introduction of Genetically Modified HNCs to Live Quagga, Zebra, and Unionid Mussels and Analysis of Engraftment, Toxicity, and Challenge of Uninfected Cultures with Live Infected Mussels of all Types

In this Example, HNCs are introduced (engrafted) to live mussels with several objectives: 1) To determine if GMDNCs can engraft to live hosts and proliferate, 2) to determine if engrafted GMDNCs proliferate and display toxic effects, 3) to determine the “host range” or specificity of GMDNCs from quagga or zebra mussels to cross-engraft or engraft to unrelated unionid species, 4) to determine if GMDNCs can travel from host-to-host by proximity, as with wild-type HN, 5) to determine if GMDNCs can be propagated and expanded both in vivo and in vitro, and 6) to determine if superior GMDNCs can be “evolved” by passage through host colonies. Methods to be used in this example will follow reports such as Elston et al. (Dev. Comp. Immunol., 12, 719-727, 1988) and Mateo et al. (J. Fish Dis., 39, 913-927, 2016).

Example 3.1. Determine if GMDNCs can engraft to live hosts and proliferate. In vitro cultured GMDNCs will be collected in their growth medium, pelleted by centrifugation, and resuspended at different concentrations for injection into live quagga and zebra mussels. Inoculated mussels will then be returned to their separate tanks for continued culture. At various intervals, hemocytes will be extracted as described in Example 1 for analysis and quantification. The method of optimal harvest and quantification will be determined in the course of experiments in Example 1 and using procedures described in Elston et al. (Dev. Comp. Immunol., 12, 719-727, 1988) and other reports. It is predicted that there will be a dose-dependent effect on HNC load, and that with time, the number of cells with GMDNC phenotype will increase.

Example 3.2. Determine if engrafted GMDNCs proliferate and display toxic effects. Inoculated mussels will be monitored on a daily basis and the number of dead animals and animals displaying signs of illness will be recorded. It is predicted that animals injected with the highest initial doses of GMDNCs will be the sickest and that death will increase with time.

Example 3.3. Determine the “host range” or specificity of GMDNCs from quagga or zebra mussels to cross-engraft or engraft to unrelated unionid species. HNCs will be examined in mussels “cross” engrafted with either quagga or zebra GMDNCs and determine the relative success of engraftment in dreissenid and non-dreissenid mussel types. It is expected that GMDNCs will engraft better in the species from which they were derived. The similarity between dreissenid mussels suggest that they may cross-engraft, but it is expected that non-dreissenids (i.e. unionid) will not permit engraftment of GMDNCs from either quagga or zebra mussels.

Example 3.4 Determine if GMDNCs can travel from host-to-host by proximity. If GMDNCs engraft after direct injection of cells, the inoculated mussels will be relocated at mid-infection into non-inoculated mussel colonies. The latter will be cultured for several months and assayed at regular time intervals. Alternatively, water from inoculated mussel cultures will be transferred to naïve cultures and monitor for engraftment, sickness, and death, as described in Example 3.2. It is expected that GMDNCs will infect all dreissenid mussels but will not engraft to non-dreissenids in a shared environment, even if they could engraft after direct injection.

Example 3.5 Determine if GMDNCs are better propagated and expanded in vivo or in vitro. It will determined whether in vivo sourced cells are better suited than in vitro cultured cells to generate the large numbers of GMDNCs that will be required for future inoculation of quagga and zebra mussels in target waterways. Thus, at various times post-inoculation, the number of GMDNCs produced will be counted and compared in live animals compared to the growth rate and expense of expanding the cells in vitro. The capacity of in vivo vs in vitro cultured GMDNCs to engraft and induce toxicity throughout a cultured colony will also be compared. The costs and features of GMDNCs produced by both methods will be weighed to determine the best method for large-scale production of GMDNCs for use in the field.

Example 3.6 Determine if superior GMDNCs can be “evolved” by passage through host colonies. It is possible, if not likely, that serial inoculation in a laboratory setting might result in GMDNCs displaying superior properties of mussel-to-mussel transmission, more rapid growth and better survival. It may also be possible to evolve GMDNCs able to cross-inoculate both dreissenid species if they are not capable of doing so in Example 3.4. This would be accomplished by inoculating target mussels with a relatively large dose of cells introduced into the water, allowing early stage engraftment, and growth to a low level. GMDNCs would then be harvested and the process repeated 2-10 times. The prediction is that cells with superior properties of engraftment will enter the animal earlier, grow faster, and increase as a percentage of the total GMDNC population each time the process is repeated. Cells from the original culture will be compared to an equal number of cells from each round of harvest and used to inoculate individual colonies to directly compare the properties of each passage. If a substantial change in engraftment and lethality is observed, further refinement can be performed until maximal utility is achieved.

### Example 4: Application in the Field

After completion of Examples 1-3, a stock of live somatic mussel cells will have been produced that are capable of engrafting to quagga and zebra mussels and triggering a cascade of “infection” capable of killing large populations of invasive mussels while leaving other freshwater mollusks, aquatic life, and animal, plant, and human populations unaffected. By way of example, personnel can bring frozen aliquots of these cells (for instance, in coolers) to sites of high invasive mussel density, and deliver (for instance, literally sprinkle) the contents over target mussel populations. Alternatively, syringes with plastic tips (that can enter between a mussel's shells but that cannot break human skin) can be employed to inject small doses of GMDNCs directly into individual target animals.

With time (for instance, days, weeks, or months), the GMDNCs will engraft and produce an active infection that disseminates throughout the local population, killing infected mussels at it progresses. If there is appreciable current in the waterway, it will be useful in some instances to focus the initial infection on upstream mussels such that HN cells produced and released by the initially infected specimens are swept downstream onto nearby mussels. Like HN infections that occur within wild-mollusk populations, the impact of this strategy on invasive mussels is predicted to be devastating.

As will be understood by one of ordinary skill in the art, each embodiment disclosed herein can comprise, consist essentially of or consist of its particular stated element, step, ingredient, or component. As used herein, the transition term “comprise” or “comprises” means includes, but is not limited to, and allows for the inclusion of unspecified elements, steps, ingredients, or components, even in major amounts. The transitional phrase “consisting of” excludes any element, step, ingredient, or component not specified. The transition phrase “consisting essentially of” limits the scope of the embodiment to the specified elements, steps, ingredients, or components and to those that do not materially affect the embodiment. As used herein, a material effect would cause a measurable decline in the population of a target species, such as quagga or zebra mussels, over a period of weeks or months, for instance when a composition including GMDNC(s) is applied to that population.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and claims are approximations that may vary depending upon the desired properties sought to be obtained by the present embodiment. At the very least, and not as an attempt to limit the application of the doctrine of equivalents to the scope of the claims, each numerical parameter is to be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing embodiments of the invention (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating that any non-claimed element is essential to the practice of the invention.

Groupings of alternative elements or embodiments of the invention disclosed herein are not to be construed as limitations. Each group member may be referred to and claimed individually or in any combination with other members of the group or other elements found herein. One or more members of a group may be included in, or deleted from, a group for reasons of convenience and/or patentability. When any such inclusion or deletion occurs, the specification is deemed to contain the group as modified thus fulfilling the written description of all Markush groups used in the claims.

Certain embodiments of this invention are described herein, including the best mode known to the inventor(s) for carrying out the invention. Variations on these described embodiments will become apparent to those of ordinary skill in the art upon reading the foregoing description. The inventor(s) expects skilled artisans to employ such variations as appropriate, and the inventor(s) intend for the invention to be practiced otherwise than specifically described herein. Accordingly, this invention includes all modifications and equivalents of the subject matter recited in the claims as permitted by applicable law. Moreover, any combination of the above-described elements in all possible variations thereof is encompassed by the invention unless otherwise indicated herein or otherwise clearly contradicted by context.

Furthermore, references have been made to patents, printed publications, journal articles, sequence database entries, and other written text throughout this specification (referenced materials herein). Each of the referenced materials are individually incorporated herein by reference in their entirety for their referenced teaching. For sequence database entries, each entry is incorporated including all information available publicly for that accession number as of the filing date of the application in which reference to the accession number is first included.

It is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Ed. Anthony Smith, Oxford University Press, Oxford, 2004).

1. An engineered disseminated neoplasia (DN) cell (DNC) from a Genus Dreissena mussel.

2. (canceled)

3. The engineered DNC of claim 1, which comprises one or more of:
a knock out (deletion) mutation of p53 or another cell cycle regulating factor;
a construct providing expression of SV40 Large-T antigen (Tag);
a construct providing over expression of telomerase reverse transcriptase (TERT) or another immortalizing protein; or
an immortalization mutation introduced using a carcinogenic agent.

4. The engineered DNC of claim 3, in which:
the knock out (deletion) mutation is generated using a clustered regularly interspaced short palindromic repeats (CRISPR)/Cas9 targeted mutation system;
the expressed Tag is expressed from a nucleic acid sequence comprising the sequence of SEQ ID NO: 8; or
the over expressed TERT protein is expressed from a nucleic acid sequence comprising the sequence of SEQ ID NO: 5.

5. The engineered DNC of claim 4, in which: the knock out (deletion) mutation is in p53 and is generated using a CRISPR/Cas9 guide RNA (gRNA) target sequence selected from SEQ ID NOs: 18-27.

6. The engineered DNC of claim 1, which is:
(a) a quagga mussel DNC and which is capable of selectively infecting Genus Dreissena mussels in a mixed population; or
(b) a zebra mussel DNC and which is capable of selectively infecting Genus Dreissena mussels in a mixed population.

7. The engineered quagga mussel DNC of claim 6(a), which is capable of selectively infecting quagga mussels in a mixed population.

8. (canceled)

9. The engineered zebra mussel DNC of claim 6(b), which is capable of selectively infecting zebra mussels in a mixed population.

10-13. (canceled)

14. An isolated immortalized Genus Dreissena mussel cell.

15. The isolated immortalized mussel cell of claim 14, which is a quagga mussel cell or zebra mussel cell.

16. The isolated immortalized mussel cell of claim 14, which comprises one or more of:
a knock out (deletion) mutation of p53 or another cell cycle regulating factor;
a SV40 Large-T antigen (Tag) expression construct;
a TERT over expression construct;
a naturally occurring mutation giving rise to its immortalization; or
an immortalization mutation introduced using a carcinogenic agent.

17. The isolated immortalized mussel cell of claim 16, in which:
the knock out (deletion) mutation is generated using a CRISPR mutation system;
the expressed Tag is expressed from a nucleic acid sequence comprising the sequence of SEQ ID NO: 8; or
the over expressed TERT protein is expressed from a nucleic acid sequence comprising the sequence of SEQ ID NO: 5.

18. The isolated immortalized mussel cell of claim 17, in which: the knock out (deletion) mutation is generated using a CRISPR/Cas9 guide RNA (gRNA) target sequence selected from SEQ ID NOs: 18-27.

19. The isolated immortalized mussel cell of claim 15, which is capable of selectively infecting Genus Dreissena mussels in a mixed population; and
(a) is a quagga mussel cell, or
(b) is a zebra mussel cell.

20. The isolated immortalized quagga mussel cell of claim 19(a), which is capable of selectively infecting quagga mussels in a mixed population.

21. (canceled)

22. The isolated immortalized zebra mussel cell of claim 19(b), which is capable of selectively infecting zebra mussels in a mixed population.

23-24. (canceled)

25. A method of killing a Genus Dreissena mussel, comprising infecting the mussel with the engineered disseminated neoplasia (DN) cell (DNC) from a Genus Dreissena mussel of claim 1, or an isolated immortalized Genus Dreissena mussel cell.

26. The method of claim 25, in which:
the engineered DNC is a quagga DNC or the isolated immortalized cell is a quagga mussel cell, and the Genus Dreissena mussel being killed is a quagga mussel; or
the engineered DNC is a zebra DNC or the isolated immortalized cell is a zebra mussel cell, and the Genus Dreissena mussel being killed is a zebra mussel.

27. (canceled)

28. A method of controlling a population of invasive, undesirable mussels comprising introducing to the population the engineered disseminated neoplasia (DN) cell (DNC) from a Genus Dreissena mussel of claim 1, or an isolated immortalized Genus Dreissena mussel cell.

29. The method of claim 28, wherein:
the invasive, undesirable mussels are Genus Dreissena mussels, and
the engineered DNC is a quagga mussel DNC or the isolated immortalized cell is a quagga mussel cell, or
the engineered DNC is a zebra mussel DNC or the isolated immortalized cell is a zebra mussel cell; or
the invasive, undesirable mussels are quagga mussels, and the engineered DNC is a quagga mussel DNC or the isolated immortalized cell is a quagga mussel cell; or
the invasive, undesirable mussels are zebra mussels and the engineered DNC is a zebra DNC or the isolated immortalized cell is a zebra mussel cell.

30-32. (canceled)

33. The method of claim 28, wherein the population of invasive, undesirable mussels is in a natural or constructed waterway or body of surface water.

34-38. (canceled)
